SEQUENCE LISTING (45 sequences)

SEQ ID NO: 1 - DNA, 1253 bp, Ashbya gossypii
  misc_feature: Oligo 19
gatctccaagcctttaaatagcgcacggtaacccataaccgaaacctcatgccgcatctctcgcagcaggtcgcctatcagatatattgtacgctctgtgtctcttgcgcggggcacactctggagccgtgtgcgcaatagctccagcggcgcaatagtagttgcagccaatatccgcgcaaatgctccacacactagtgggtttgccacaggtagccgggatgccaagggcgagttatcacgcagtgcttcatagccggaaaagtacaccacgttagccggcaccgccattacgagcgtaatccccagccctcgccacagagtcggcagaccctctagttgggctatcttcctgagcccctccagagttccctgcagcctcgcagcaggctctcggcagccgacgtttgcaaagcactcatcctgccaaaacactttccctgccggtttcgacagctgtcctgtacatgtacaacttggaagcatttcttgctgttgaagccgcacgcggaccacgtccagtggcgtgagaaacagtgacgtgaccagcgagccagcagaggcgcttaccatgcgttncttcagcgtaaccttctttccgggttccttactcatggccccaaatttgcacctttcagcgctaaccccgtccaatagtactgcctcttcctgttagcctatcttgccgctcgagagtaacttatgttttgatcgaagctttcttgggggcgattattcgctgtgtatgggacttagttttactatgcgctaaaatgttgtcggggcccgaaagccgttgtacggcgtcagcgttgatgtgcaccccgtggctatttaataatatactatataaatactaaaactattaagtattaatcatgatgatgtggaatcagtatttcagtaagatgtagcatcagagttgtccaatgggacctccgggctttcctcgtatagtcttaaaaccatggaggttatccgtttcagctggaattgtaatagttcgtcgacggttgccatattaatctgggtcttctccccgcgctggaacaaagacatgtcaaattcatcctcctcccagttcatgtactcgtcaatgctatagcagtcgtgagtcagggtactcttcggtgaacaatatgtattggaacattttctatccattggttaaccttctcgcggtggggcatgttcagcgctgcgcgcggattaaaataggcaggaaagctcgactgtggaacatcgtagggatc

SEQ ID NO: 2 - protein, 203 aa, Ashbya gossypii
  misc_feature: Oligo 19
KEPGKKVTLKXRMVSASAGSLVTSLFLTPLDVVRVRLQQQEMLPSCTCTGQLSKPAGKVFWQDECFANVGCREPAARLQGTLEGLRKIAQLEGLPTLWRGLGITLVMAVPANVVYFSGYEALRDNSPLASRLPVANPLVCGAFARILAATTIAPLELLRTRLQSVPRARDTERTIYLIGDLLREMRHEVSVMGYRALFKGLEI

SEQ ID NO: 3 - DNA, 2020 bp, Ashbya gossypii
  CDS (112)..(294)
aatccccctatattcaacaacagtccgcagtatttcccagatctgaactatttatctccatggcacagttattcaaacggtgtcggcaccccaataccaattaacagttccatgattgggaacccaaacctttccaacgtccccatgaccaagacgtacgatccctacgatgttccacagtcgagctttcctgcctattttaatccgcgcgcagcgctgaacatgccccaccgcgagaaggttaaccaatggatagaaaatgttccaatacatattgttcaccgaagagtaccctgactcacgactgctatagcattgacgagtacatgaactgggaggaggatgaatttgacatgtctttgttccagcgcggggagaagacccagattaatatggcaaccgtcgacgaactattacaattccagctgaaacggataacctccatggttttaagactatacgaggaaagcccggaggtcccattggacaactctgatgctacatcttactgaaatactgattccacatcatcatgattaatacttaatagttttagtatttatatagtatattattaaatagccacggggtgcacatcaacgctgacgccgtacaacggctttcgggccccgacaacattttagcgcatagtaaaactaagtcccatacacagcgaataatcgcccccaagaaagcttcgatcaaaacataagttactctcgagcggcaagataggctaacaggaagaggcagtactattggacggggttagcgctgaaaggtgcaaatttggggccatgagtaaggaacccggaaagaaggttacgctgaaggaacgcatggtaagcgcctctgctggctcgctggtcacgtcactgtttctcacgccactggacgtggtccgcgtgcggcttcaacagcaagaaatgcttccaagttgtacatgtacaggacagctgtcgaaaccggcagggaaagtgttttggcaggatgagtgctttgcaaacgtcggctgccgagagcctgctgcgaggctgcagggaactctggaggggctcaggaagatagcccaactagagggtctgccgactctgtggcgagggctggggattacgctcgtaatggcggtgccggctaacgtggtgtacttttccggctatgaagcactgcgtgataactcgcccttggcatcccggctacctgtggcaaacccactagtgtgtggagcatttgcgcggatattggctgcaactactattgcgccgctggagctattgcgcacacggctccagagtgtgccccgcgcaagagacacagagcgtacaatatatctgataggcgacctgctgcgagagatgcggcatgaggtttcggttatgggttaccgtgcgctatttaaaggcttggagatcactttatggagggacgtgcccttcagcgcaatctattggggaacatacgagttctgtaaaacccagttctgggcccgccatgccgcaacccataatgcatcaaactgggaccatttcatcggcagttttgcctgcggtagcatgggcggtgctgttgcagcacttttgacacatccttttgatgtgggcaagacccgcatgcagattgcgattgccagtccacagcagctaactgtggggggaaaagctacgaaaactgatgactcaagaggcatgttctcatttttgaatgccattaggaaatcagaaggtattagagcgctatataccggcctattacctagggtgatgaagattgcaccaagttgcgccataatgatctcgacttatgaactgtcgaagaagttcttcactagttgaactttgtttcttcctgtatataccaagtaattcacactgttgatacacgatacagcatttttcttaaaaccctgtgatatacgcttgctgtagtacacgcccaaacggtgaggtctataattttacatgtccgcgagatggtattggatcggcagaattttagagctgccgctgtagcccatccgtcg

SEQ ID NO: 4 - protein, 61 aa, Ashbya gossypii
  misc_feature: Oligo 19
MIGNPNLSNVPMTKTYDPYDVPQSSFPAYFNPRAALNMPHREKVNQWIENVPIHIVHRRVP

SEQ ID NO: 5 - DNA, 1495 bp, Ashbya gossypii
  misc_feature: Oligo 24
gatcgccacctggtccctgttcagcgcgccggacgtctgctgcgcctccagcgggtccaggaacttcagcatcgcgggctcgatctgcgaaaagcacttgtacagcgggtagaagaggccacgcccgtcctggatccagtcgaagtactgtggcatgtgcgacaccaccacgtccctagaaaacgtccccttcagcgacaccttcagatcctgcagcaggttgaccaatcgcaacaggtcctgcttgatctcctccgtgctcaggttcttgtttccgattcgcagcatcacatcctccagtgagtcagctccgggccctagtgtggtcgctaaggggggcgagataaacgcgttaggtacctgtatcatgctgcggacaccacctcactttgaaatattacaactgtgctatcagctgccctttataagcagcgctgtctccatacaattgcgctgattttatgtagttgatgcgtgtaatctgctttttccctattgagacgtcagcatctcgtagcaaggctccgtgtgcttacttaagaaaagatggaatcgatggaatcgatgaaactaaaacattttatcaaatatcagttctatatatatgtagttattgggagataatacgggtgaatctggggcatgtcttaaggcattggggcacgggccgtgtcctccagcacatgctttatgacctcttcatttaggcgaagatttccgccctcgatatcgcggggaatgtctgcggcctgtggagcggaagcctcttcctgggcaggagctggagacgcctcgggcttcgcaggctccgacgcgtgttcgttcaatagcttgaacaactcgctgtcctcgtcacggtataggtcggcaaacttgccggcctcgatgacggagccgtcattgccgagaaccacgatgttctcggagcggcgtatggtgctgaggcggtgggcgatgctcacgatggtgatctccttgctgcgcattagcctgccgagggtgtagttgatggcgccctcgctttcaacgtccaacgccgaggtggcctcgtcgaggattaggattttcggtttcttgatgagcgcgcgggcgatggcaatgcgctggcgctggcccccgctgagtagggcgcctctaggcccaatcacggtgtcatagccgtccgggaacttggtgatgaagttatggcagaagcacttcttggcaactgcgcgtatctcatccatggagggcgtgtaggaaaggccgtaggtgatgttgtctctgatggtcccggacatcaagaccggctcctgctggacaacgccaagatgttttctgagcgacttggcgcttaccttggatatgtcctgaccgtctatgaggatggtgccgctgttggcatcngtaaaaccgtaggaggagggacgcaatggtggacttgccccgcccggagggcccgacgatgcagacgttggagcccggggttatcgtgaggcggagtttcttcaagatc

SEQ ID NO: 6 - protein, 36 aa, Ashbya gossypii
  misc_feature: Oligo 24
ILKKLRLTITPGSNVCIVGPSGRGKSTIASLLLRFY

SEQ ID NO: 7 - protein, 225 aa, Ashbya gossypii
  misc_feature: Oligo 24
GPPGGASPPLRPSSYGFTDANSGTILIDGQDISKVSAKSLRKHLGVVQQEPVLMSGTIRDNITYGLSYTPSMDEIRAVAKKCFCHNFITKFPDGYDTVIGPRGALLSGGQRQRIAIARALIKKPKILILDEATSALDVESEGAINYTLGRLMRSKEITIVSIAHRLSTIRRSENIVVLGNDGSVIEAGKFADLYRDEDSELFKLLNEHASEPAKPEASPAPAQEE

SEQ ID NO: 8 - DNA, 4118 bp, Ashbya gossypii
  CDS (820)..(3081)
ctgtcacagcaagacctggcttcccgtcacgctatggctcgcctgatcatctcatctcgcctgcgcatcgagcgcagctcaaaatttttaccattaccccggccagtacacatattacataatcaccagtgtccagggattacccggacatcaaacatcacagagaacgaacccaatgaaatgcaggaaaagctgctcagcgccggtgcccggcggccattaagcgtgcacctacagccacttcactccaggccacacactccacgcctccccgcaagtaaacacgatgtctgcctggagaaaagccggtttgacctacaatagctacctggccgttgcggccaggaccgtgcgcgcggctctgaagaaggagctccagagccctgccgtcctcaaccgatctgtgacggaggcaaaggtcatcgactacgcctccaagggctccgccgcggaggccgtgccgctacggaagtgagcagcacgcgtgctcttctcgttgaactgctggcacgcagcctgggccctgttggcggcaagcgggcggatggagacgcccagcacaggccctttgacgggctgtgctggtggcatgtgtgtagttcgactatataaactctcgttctctaatgataatgacagaaggtcgattgggcgatttcggtgctgcgaacggggacgttgtgcaggggcaaaggcggttttcgcgggcaagcgaagaggtgcgttttccgcaagcttgcttgtgtccccgcccaaggcaagacacagtctactcgcatcagttttggcagagcgtgttgctacaggattccaggcgctatgtggagagcgataaggccctttggggggcttggccctgtagcggtgttacagaggctaccgggcccgcggcacaatgtcgccgtgaggcttcaggtgatgtccagggccagatgcggctggcagtgtgtgcgtgcgacatctcgactgacagtgccggtgtgcaggcaaagccgggcgtacagcgctgcccgtgtgggcggcgagggaacgccttcgccgcgggacaaggagcatgagcaggagcgcaaagttgaaaagcgaccggcggagccgtcgcgcgcgcaacccgcaaagccagccggctggcacgaggtgttccggctcttgcgattggtgaagcccgactggaagttgttgcttgcggcgttggggctcctcacgatatcttgctcgattgggatggcggttccgaaggttattgggcttgtgctagatgccacgaaagacgcagtcgcacaggcagaaaagaacgaaacgggcgcgccgatgtctttgctagacatgccgcctatcatcgggaacttcacgctagtcgacgtgcttgctgcgtttgcgggcgcgttgctcgtgggctccgccgcgaacttcgggcggatgttcctcttgaaaatgcttggtgagcgtttggttgcgcggctacgtgcgcaggtcatcaagaagacgttgcatcacgacgcggagtttttcgacaggaacaaggtcggcgacttgatatcgcggcttggttccgacgcctacattgtatcgcgctcgatgacgcagaacatttcggacggggttaaagccgcgctctgcggaggcgtcggtgtgggcatgatgttccacatttcgagcacattgacttctgcaatgataatgtttgcccctcccctcctcatcgccgccaccatctacggcaagcgcattcgcgccatctcgcgcgagctccagcaatctacaggtaacttgacgcgggtgtcagaggagcagttcaatggcatcaagaccatcaagtcattcgtcgcggagggaaaggagatgcggagatacaacacggccgtgcggaggctcttcaacgttgcaaagagggaaggtataacgagcgcgacgtttttcagcggtacgaatgtgttgggcgacgtgagttttttgcttgttttggcgtatgggtcgcacctcgttttgcagggcgaactatcccttgggagcttgacggcgttcatgctctacactgagtacactggaagctcggttttcggtttatcgaacttttactcagagttgatgaagggcgctggtgctgcttcgcgcttgttcgaattgacagactacaagaactccgtgccgtctaccgttggccagacgtttgtgccgcgcgatggtcgcatcgagttcaaggacgtgtccttcagctaccccacgcggccgacgagccagatcttcaagaaactccgcctcacgataaccccgggctccaacgtctgcatcgtcgggccctccgggcggggcaagtccaccattgcgtccctcctcctacggttttacgatgccaacagcggcaccatcctcatagacggtcaggacatatccaaggtaagcgccaagtcgctcagaaaacatcttggcgttgtccagcaggagccggtcttgatgtccgggaccatcagagacaacatcacctacggcctttcctacacgccctccatggatgagatacgcgcagttgccaagaagtgcttctgccataacttcatcaccaagttcccggacggctatgacaccgtgattgggcctagaggcgccctactcagcgggggccagcgccagcgcattgccatcgcccgcgcgctcatcaagaaaccgaaaatcctaatcctcgacgaggccacctcggcgttggacgttgaaagcgagggcgccatcaactacaccctcggcaggctaatgcgcagcaaggagatcaccatcgtgagcatcgcccaccgcctcagcaccatacgccgctccgagaacatcgtggttctcggcaatgacggctccgtcatcgaggccggcaagtttgccgacctataccgtgacgaggacagcgagttgttcaagctattgaacgaacacgcgtcggagcctgcgaagcccgaggcgtctccagctcctgcccaggaagaggcttccgctccacaggccgcagacattccccgcgatatcgagggcggaaatcttcgcctaaatgaagaggtcataaagcatgtgctggaggacacggcccgtgccccaatgccttaagacatgccccagattcacccgtattatctcccaataactacatatatatagaactgatatttgataaaatgttttagtttcatcgattccatcgattccatcttttcttaagtaagcacacggagccttgctacgagatgctgacgtctcaatagggaaaaagcagattacacgcatcaactacataaaatcagcgcaattgtatggagacagcgctgcttataaagggcagctgatagcacagttgtaatatttcaaagtgaggtggtgtccgcagcatgatacaggtacctaacgcgtttatctcgccccccttagcgaccacactagggcccggagctgactcactggaggatgtgatgctgcgaatcggaaacaagaacctgagcacggaggagatcaagcaggacctgttgcgattggtcaacctgctgcaggatctgaaggtgtcgctgaaggggacgttttctagggacgtggtggtgtcgcacatgccacagtacttcgactggatccaggacgggcgtggcctcttctacccgctgtacaagtgcttttcgcagatcgagcccgcgatgctgaagttcctggacccgctggaggcgcagcagacgtccggcgcgctgaacagggaccaggtggcgatcttcaacctgatcgaccaggtgtccgagctcgtgctgagcctgaagccgctgttccagtcggtgaagaacctattcgacacggcgctggagttcaatgagatcttcaaggaccacatgaactcgctactggaggagatcgaaggaaacatgaagaagtgcctcgcgctgcaccaggactgcttcgcgtcgcccgtgagacaccctccgtccttcacgttggaccagctcgtggagcttatatcctcatcgtcaaacaaccagcggctgcagatgccgaccttcaatcccctggaaaagcgcatctaccagggattactgcgagctaagagaatgctattgtgccgatccagacatcgctgaagagacgtcctgaaaacccgcatccca

SEQ ID NO: 9 - protein, 754 aa, Ashbya gossypii
  misc_feature: Oligo 24
MWRAIRPFGGLGPVAVLQRLPGPRHNVAVRLQVMSRARCGWQCVRATSRLTVPVCRQSRAYSAARVGGEGTPSPRDKEHEQERKVEKRPAEPSRAQPAKPAGWHEVFRLLRLVKPDWKLLLAALGLLTISCSIGMAVPKVIGLVLDATKDAVAQAEKNETGAPMSLLDMPPIIGNFTLVDVLAAFAGALLVGSAANFGRMFLLKMLGERLVARLRAQVIKKTLHHDAEFFDRNKVGDLISRLGSDAYIVSRSMTQNISDGVKAALCGGVGVGMMFHISSTLTSAMIMFAPPLLIAATIYGKRIRAISRELQQSTGNLTRVSEEQFNGIKTIKSFVAEGKEMRRYNTAVRRLFNVAKREGITSATFFSGTNVLGDVSFLLVLAYGSHLVLQGELSLGSLTAFMLYTEYTGSSVFGLSNFYSELMKGAGAASRLFELTDYKNSVPSTVGQTFVPRDGRIEFKDVSFSYPTRPTSQIFKKLRLTITPGSNVCIVGPSGRGKSTIASLLLRFYDANSGTILIDGQDISKVSAKSLRKHLGVVQQEPVLMSGTIRDNITYGLSYTPSMDEIRAVAKKCFCHNFITKFPDGYDTVIGPRGALLSGGQRQRIAIARALIKKPKILILDEATSALDVESEGAINYTLGRLMRSKEITIVSIAHRLSTIRRSENIVVLGNDGSVIEAGKFADLYRDEDSELFKLLNEHASEPAKPEASPAPAQEEASAPQAADIPRDIEGGNLRLNEEVIKHVLEDTARAPMP

SEQ ID NO: 10 - DNA, 1002 bp, Ashbya gossypii
  misc_feature: Oligo 109
gatcggagccgattaaaacggcacaagcttgggagctgatcgaactactttcacaaaatgatgtcgtgaagtatggagatatcgttttcaggccgttgtttaagtcttctccagaggccgggttgttggagttagagaaaaatgggctgatcacaatcagccgcaatagaggagtgttgcaagatatccggcccgctaaaccattgtttaaagccgcattcagctacctgttacaggacaaggacctgtccatcgtcctccgaacaggttactacttgcgactaatcgcgtttgaaactgggcggatcaaaaagtgggaggaggaattgcgtctattggcaaaggttagtgaccaaagaatatgcaagagtcggttgaattacttagcaagcaaaatagacgccagtagtggggtaattaacagttgtgaggacaaggtaaaagaaatgtcaaagcgtatttagaaacgctcgcatcaataagaccccattttgccacgatctggcaagatgaactaaatagcgataatcacactaggtctcattagtttagcattccagctgcacgggctttagtttagtacctcaattccaaaaacggcactatcttgtccgagcgctggtgaatatccttgccgtatgtgaaattatccagggttgatgtagttggccggagctaccagtaattgttggcctatgcacgggccgctattttccgtaacataatccagagatccgttaacaggcaacatgaactgattgattaattttaatgaacttttttttttggctttgaaaggcctaaaattggatcatctacattccctgacgatataggatggtaaaagccctaccaaccaagaaattctgatccctttgacaaatcccttataacatagatattagaatataatcaaccccattgtgtgacaactcttctccaaaaataattggtctttcacacaattaaagattggcttcaaggtagctccacggtgagatc

SEQ ID NO: 11 - protein, 147 aa, Ashbya gossypii
  misc_feature: Oligo 109
KTAQAWELIELLSQNDVVKYGDIVFRPLFKSSPEAGLLELEKNGLITISRNRGVLQDIRPAKPLFKAAFSYLLQDKDLSIVLRTGYYLRLIAFETGRIKKWEEELRLLAKVSDQRICKSRLNYLASKIDASSGVINSCEDKVKEMSK

SEQ ID NO: 12 - DNA, 4077 bp, Ashbya gossypii
  CDS (502)..(2919)
cctgtaccgggtgcggtgccagatacatcgcaatccccttcgtgctgttgccatgcgggacacaagcaggtcccattcccatctgcccacctgcacccgcggcgagtaatgcttctcccccccgtagttcgctccaccgctcgatccgccacgtgaaccggccctcgtactcggtctcttctccttctagcggcggcaaaacctcctcgaacgtcttgccgagctccaccgcctccatgtttgtttcccgggtttctggcacatccattactatcttgatatagttcgatgttgatactgcctgggtccagcctactatccaagtcctggtttgttcatcgtgtgtagccctgtagagagtgaatttcaaggggcaggtaacattgccggacgcctacacccgtggcttctgaagaccatcgcaaaaggtattcagaactttgcaactagtaccagcgatacaaggccggtgacagcagaagtaacgtcccgagaaggccaatgatgtttctggagatgcagcgagcgttcatgctacacgggcggcgagcagtgacgagaagtgcggtcggcgtgcggtacatctcggaggacatccagcagaaggacgcacaggcgggcgagaaggcgacggcgacggcgacgggggttatctacaagtcggacgaggaaacgctgatgtatttcgacaacgtatacccgcgggcgacgtcgttgtggcggccgacgcagtggtacaacattctgctgtcgaaccagtcgcgggaagcggtgagggagaagatcatgcggttcgcaagcccggcaagcaacccggtgcacggactggagctgcggtcgacgatcccgatcaagcgggacggcggggtgtttgcgacgttccgggtgccgcgtgagtacacgagggcgcaggtgaacgcgttgatccaggcgaccacgcagcaggagtcgtcgaagtcgctgctggcggcgtttacgcgggctgcagcgttcccggtgaagggcgtgccgtggatcgaggacctgaagcggctgccgaacaacgtggtgcgcgtggaggtggagggcccggcgctgtcggaggaggagttgtactcgctgttccggcggtacggcacgattctggacatctaccccgcgggcaagaacggctatgcgacgatccgctaccgctcgttccggggcgcgatctgcgcgaagaactgtgtgtcggggatcgagatcaacggctcgacgctgcatgtcaagtttgagcctgtagtccgcgcgcacgcgatccgcgacttcttcgtgaaccacccgcgcattgccattcccctgctgattgccctgttatccatctgtgctgtgctgatcttcgaccctatccgggagttttccattgagcagaagatcacgcgcatgtacacactgtcgcgggacaacttcgtggtgaaaagcatcctgcgcctcacctcctatacggtctcatctgtgaagcatctctggggctacgacgatgaccagccagagaaacgacagttgtggcaggagcgcgtggagaaggtgaacgacctgaagatgtggttagaggaaaacaacaacactttcgtcgtggtgaccggtcctcgcggctctggcaagcatgagctcgtgatgcagcacactctgcatgaccgtcctaatgtgctatatctggattgtgatactttgatcaagtcgcgtactgattccaagtttttgaggaatgccgcccaccagattggctatttcccaatattcccgtggttgaattccgtcacgactctggttgacctggctgttcagggcctgaccggccagaagtccgggctatctgagtcaaaggaaacgcaattccggaatatgctgaacactgctatgatgtctattaggcacattgctttgtccggttataaggcgactctgcactctggcgatgatgttacgactgtgaaggaggaggactacttgcaacagcatcctgagcgcaagccggttattgtaatagacaggttcagtaacaaggctgagataaacggttttgtgtacaaagaactcgcagattgggcgtccatgcttgttcagatgaacatcgcgcatgttatattcctcaccgagtctgtatctccaaaccagttgttggcagaggctcttccaaatcaagttttcaagttcctgtttctctcagatgcgtcgaaggactcagcgaggagctacgtgctttcacagttatacccttctagcccagcgtattctgaaaaaatgccagcagcagatgcagatgccaatgaggagtataggaaggagatagaccgtgcattggagcctatcggcggtagaatgttggacctacaagcgtttgtccgcagagtgaaatcaggggaggagccgtctgaagcattggaaaagatggtggagcaagcatcagagcagatcactcaaatattcttaagtgagagatcggagccgattaaaacggcacaagcttgggagctgatcgaactactttcacaaaatgatgtcgtgaagtatggagatatcgttttcaggccgttgtttaagtcttctccagaggccgggttgttggagttagagaaaaatgggctgatcacaatcagccgcaatagaggagtgttgcaagatatccggcccgctaaaccattgtttaaagccgcattcagctacctgttacaggacaaggacctgtccatcgtcctccgaacaggttactacttgcgactaatcgcgtttgaaactgggcggatcaaaaagtgggaggaggaattgcgtctattggcaaaggttagtgaccaaagaatatgcaagagtcggttgaattacttagcaagcaaaatagacgccagtagtggggtaattaacagttgtgaggacaaggtaaaagaaatgtcaaagcgtatttagaaacgctcgcatcaataagaccccattttgccacgatctggcaagatgaactaaatagcgataatcacactaggtctcattagtttagcattccagctgcacgggctttagtttagtacctcaattccaaaaacggcactatcttgtccgagcgctggtgaatatccttgccgtatgtgaaattatccagggttgatgtagttggccggagctaccagtaattgttggcctatgcacgggccgctattttccgtaacataatccagagatccgttaacaggcaacatgaactgattgattaattttaatgaacttttttttttggctttgaaaggcctaaaattggatcatctacattccctgacgatataggatggtaaaagccctaccaaccaagaaattctgatccctttgacaaatcccttataacatagatattagaatataatcaaccccattgtgtgacaactcttctccaaaaataattggtctttcacacaattaaagattggcttcaaggtagctccacggtgagatctgctaaaacagtatggaagctgctaccattaatgaagagcaagtcatctctgaaagggatttggaaaagctgccagtgtcgagaaatgtcagtagaggatgggcagttcgtaagttggcaatattatactttctattagcgtctttcgctatagggacggtgcacttattccagtctggttacatgggtgctaatagtaaatctcctcagatcggtcgctggcaactatttgcggagaattctcagggccttacaaaacggagtagatgggcgatgatcgctgaaaaacaccacgcgcccctcttagcagcggcaagtctatcaacggcagtggagcctaatgtttgcccgcctgatggcagaagatgcatgggcgaactaatcaccagattattacttcaatcactcgcgatagctcaggacctctatggtggtgagtggaagattactcagccacatccaccatacaacaacaacaaacgcagtctgtcgaactatgcatttgaaactgcatcggaactttcaactcaaataaagagtagagcagacactttggcggcgttaaatatcaatctggtgggacgcttccgcagcaatgacccaaatggccttgcgaa

SEQ ID NO: 13 - protein, 806 aa, Ashbya gossypii
  misc_feature: Oligo 109
MMFLEMQRAFMLHGRRAVTRSAVGVRYISEDIQQKDAQAGEKATATATGVIYKSDEETLMYFDNVYPRATSLWRPTQWYNILLSNQSREAVREKIMRFASPASNPVHGLELRSTIPIKRDGGVFATFRVPREYTRAQVNALIQATTQQESSKSLLAAFTRAAAFPVKGVPWIEDLKRLPNNVVRVEVEGPALSEEELYSLFRRYGTILDIYPAGKNGYATIRYRSFRGAICAKNCVSGIEINGSTLHVKFEPVVRAHAIRDFFVNHPRIAIPLLIALLSICAVLIFDPIREFSIEQKITRMYTLSRDNFVVKSILRLTSYTVSSVKHLWGYDDDQPEKRQLWQERVEKVNDLKMWLEENNNTFVVVTGPRGSGKHELVMQHTLHDRPNVLYLDCDTLIKSRTDSKFLRNAAHQIGYFPIFPWLNSVTTLVDLAVQGLTGQKSGLSESKETQFRNMLNTAMMSIRHIALSGYKATLHSGDDVTTVKEEDYLQQHPERKPVIVIDRFSNKAEINGFVYKELADWASMLVQMNIAHVIFLTESVSPNQLLAEALPNQVFKFLFLSDASKDSARSYVLSQLYPSSPAYSEKMPAADADANEEYRKEIDRALEPIGGRMLDLQAFVRRVKSGEEPSEALEKMVEQASEQITQIFLSERSEPIKTAQAWELIELLSQNDVVKYGDIVFRPLFKSSPEAGLLELEKNGLITISRNRGVLQDIRPAKPLFKAAFSYLLQDKDLSIVLRTGYYLRLIAFETGRIKKWEEELRLLAKVSDQRICKSRLNYLASKIDASSGVINSCEDKVKEMSKRI

SEQ ID NO: 14 - DNA, 1337 bp, Ashbya gossypii
  misc_feature: Oligo 163
gatcttggacttgacacagacagtggcacgttacggctggttaccattcatcctttacatgggctggtcgcacactgccaactcccccaacctgctaaaccttctctccccactcccaagtgtctaatgtatttgcaacggttccctcagtatgtgcttttgttcacatgatcctcctagggcaaggccgggacggagggaacgatagcgcgaggctaccagcctcggtcctaccacgtcgagtgtgccgcaattctagtgagcttgtatatacgtaatttgaataaataagcgttcattgttaccttatccgcctatgagttcgggaactacctgttcgctgcctttgtgaaaagtagctgacaatagcagcggtgatgcatcttccgaaacagccataggagagcaatgggccacgagctgacaagcctgcagcgtgaagtgatttccggactcacagcaggaacaatcacaacaatcgctagccacccgctcgacctgcttaaactgcgactacaattatcagcgggcaacagggctaacactacatatacaggactaatcagagacatatttgaacgccagcaatggggacgagagctatatcgtggtttaggcgttaacctgcttggaaattcagtggcatgggcgctttactttggctgttaccgatgcgcaaaggacattgcgcttcgacacttgggcaatgagtctgctacgggtatcatggaccgccgcctgccagcgcacgcatacatgcttgcagctggtagcagcgggattgcgacggcagtccttacaaaccccatatgggtcattaagactcgcataatggccacttctcgtgctggaccttacaagtcgacgtttgacggggtttataagttatatcaaactgaaggtgttctggcattctggcggggtgttgttccatcgctactcggcgtctcgcaaggagctatctactttgcgctgtatgatacattgaaattccattacctgcactctagtactgacaaggccgagcgaaggttgtcggtttccgagatcatcggcataacatgtatctctaaaatgatttccgtcacatcggtctatccatttcagctgctgaaatctaagctgcaggattttggtgcaccatccggcatcacccaacttgttcagactgtttacagtagagagggtatcagaggcttctacaggggcctatcctgctaatctcctgcgggcagttcctgctacatgcataacctttttcgtgtacgaaaatatcaaatatcgtctctaaatttgtgatttggtgcagcgcgtagcaagtatacactgactggatc

SEQ ID NO: 15 - protein, 267 aa, Ashbya gossypii
  misc_feature: Oligo 163
HELTSLQREVISGLTAGTITTIASHPLDLLKLRLQLSAGNRANTTYTGLIRDIFERQQWGRELYRGLGVNLLGNSVAWALYFGCYRCAKDIALRHLGNESATGIMDRRLPAHAYMLAAGSSGIATAVLTNPIWVIKTRIMATSRAGPYKSTFDGVYKLYQTEGVLAFWRGVVPSLLGVSQGAIYFALYDTLKFHYLHSSTDKAERRLSVSEIIGITCISKMISVTSVYPFQLLKSKLQDFGAPSGITQLVQTVYSREGIRGFYRGLS

SEQ ID NO: 16 - protein, 47 aa, Ashbya gossypii
  misc_feature: Oligo 163
ASPNLFRLFTVERVSEASTGAYPANLLRAVPATCITFFVYENIKYRL

SEQ ID NO: 17 - DNA, 1733 bp, Ashbya gossypii
  CDS (329)..(1207)
actcccccaacctgataaaccttctctccccactcccaagtgtctaatgtatttgcaacggttccctcagtatgtgcttttgttcacatgatcctcctagggcaaggccgggacggagggaacgatagcgcgaggctaccagcctcggtcctaccacgtcgagtgtgccgcaattctagtgagcttgtatatacagtaatttgaataaataagcgttcattgttaccttatccgcctatgagttcgggaactactgttcgctgcctttgtgaaaagtagctgacaatagcagcggtgatgcatcttccgaaacagccataggagagcaatgggccacgagctgacaagcctgcagcgtgaagtgatttccggactcacagcaggaacaatcacaacaatcgctagccacccgctcgacctgcttaaactgcgactacaattatcagcgggcaacagggctaacactacatatacaggactaatcagagacatatttgaacgccagcaatggggacgagagctatatcgtggtttaggcgttaacctgcttggaaattcagtggcatgggcgctttactttggctgttaccgatgcgcaaaggacattgcgcttcgacacttgggcaatgagtctgctacgggtatcatggaccgccgcctgccagcgcacgcatacatgcttgcagctggtagcagcgggattgcgacggcagtccttacaaaccccatatgggtcattaagactcgcataatggccacttctcgtgctggaccttacaagtcgacgtttgacggggtttataagttatatcaaactgaaggtgttctggcattctggcggggtgttgttccatcgctactcggcgtctcgcaaggagctatctactttgcgctgtatgatacattgaaattccattacctgcactctagtactgacaaggccgagcgaaggttgtcggtttccgagatcatcggcataacatgtatctctaaaatgatttccgtcacatcggtctatccatttcagctgctgaaatctaagctgcaggattttggtgcaccatccggcatcacccaacttgttcagactgtttacagtagagagggtatcagaggcttctacaggggcctatccgctaatctcctgcgggcagttcctgctacatgcataacctttttcgtgtacgaaaatatcaaatatcgtctctaaatttgtgatttggtgcagcgcgtagcaagtatacactgactggatctaaaagatgattaatttgtcttaagctttacaaaaaaacactttatatattgattgatattgggcaaaagattcatgaccttatgatgaaatttagtaaccgagcttggccaaatcctcagcgatctcggacttagcatcatatttagccttgacagcagcagccgttttgttgaaggccctcttcttggagtagtactcagcagatctgacctttctcttctcttccaacttggcaacaacgttctcgtacttccagccaacggtggaagacaacttacccaaggtggtgaactttctacctggcttcaatctcaaaactctaaagcctgtggaacaacaactctcttcaacttgtcgtatggaggtgggacaccctctaagacctttaatctctccatagcagccttaccacggggcagtcttgtgagcaaccatacctctgacagccttgtagaagaatctggatggggctctg

SEQ ID NO: 18 - protein, 293 aa, Ashbya gossypii
  misc_feature: Oligo 163
MGHELTSLQREVISGLTAGTITTIASHPLDLLKLRLQLSAGNRANTTYTGLIRDIFERQQWGRELYRGLGVNLLGNSVAWALYFGCYRCAKDIALRHLGNESATGIMDRRLPAHAYMLAAGSSGIATAVLTNPIWVIKTRIMATSRAGPYKSTFDGVYKLYQTEGVLAFWRGVVPSLLGVSQGAIYFALYDTLKFHYLHSSTDKAERRLSVSEIIGITCISKMISVTSVYPFQLLKSKLQDFGAPSGITQLVQTVYSREGIRGFYRGLSANLLRAVPATCITFFVYENIKYRL

SEQ ID NO: 19 - DNA, 972 bp, Ashbya gossypii
  misc_feature: Oligo 31
gatcgatattgtcggcaacttcattcctggattaatatttatgcaatcaatatttgggtacctatcgtgggcaattatttacaagtggtcaaaagattggattaaagatgagctgcctgcgcctggtttattaaacatgttgattaacatgtttttatctcccggagttgtcgatgaaaaactatatacaggccagagctttcttcaagttatccttttgctagctgctctagtttgtgttccgtggctactactttacaaaccgttgatgttgaagcgccagaacgatatagctctcagcaaaggattcagaagcctcagagaccaacgggtgcatgagattcttctggaagcacaggaaaacgcaggcgaagatatgctggttgcagattatgaaaatgaagatgaatcgtcggaggagttcaactttggtgacgttatgatacatcaggttatccacaccattgaattttgtttgaattgtatatctcatactgcatcgtatctcagattgtgggccttatctcttgcgcatgctcaactctcgactgtgttgtggtctatgaccattcagaattcgttctccgactccaaccctggttcatttttctctgtcaccaaggtggtggttttgtttgccatgtggtttgtgttgactgtttgcattttagtcttaatggaaggaacgtcggctatgttgcactcgttaagattgcattgggtggaagcgatgtccaaattcttcgagggcgaaggctatgcttacgaaccattttccttcaaggctatcaacagcgatgatgaatagtatcgtatatattaaaactagacagatggaggtagtgtcacatgtgtcaggtatagattgcccgatgaataagcatccttgaaatttagaaaaatcataggcctttagaatcgacatacaacgtctaaatatatttactattcacttcatagttcatcgtgatc

SEQ ID NO: 20 - protein, 263 aa, Ashbya gossypii
  misc_feature: Oligo 31
IDIVGNFIPGLIFMQSIFGYLSWAIIYKWSKDWIKDELPAPGLLNMLINMFLSPGVVDEKLYTGQSFLQVILLLAALVCVPWLLLYKPLMLKRQNDIALSKGFRSLRDQRVHEILLEAQENAGEDMLVADYENEDESSEEFNFGDVMIHQVIHTIEFCLNCISHTASYLRLWALSLAHAQLSTVLWSMTIQNSFSDSNPGSFFSVTKVVVLFAMWFVLTVCILVLMEGTSAMLHSLRLHWVEAMSKFFEGEGYAYEPFSFKAI

SEQ ID NO: 21 - DNA, 4054 bp, Ashbya gossypii
  CDS (623)..(3253)
ccgtgcagtacatcttgcgcattgccagtataggtcacccaccactgtcgtcgcctgcttctcgtatacccactgttccacaacatacagctcgtacccaccaagtgccacttcttggtacgaacaatcctccaaagcgctctgcgacctgaaaaccgcccggaagtccggcacaacgtactttatccatccgccatgtgtttcggcctcaccatcgttcaccactttgcgtgaacctcgagagttatgctgggcaatatccatatctaagctccatctactactgttcgctacctcacgggctgcctaggaagtggttcatagacgatatggagctatagggctcatcgcagtccgtcataaggcatgttcaccatcacagaaaacgatttaaaagggacacccacaacttcaaccactacaaataccagtaaggtatgcgctaggaagcagttggagttactagctgttaggagacattgaaacgagtatctaaagattttgacagtggggattctgtgtgcattggtgttcatgcacgtagtgactaagtagtggctgcatacatagggtacacagactgacagggtggctgacaaaagaacagtaacttgagatcgtgatgttcctatcaatgcgggccgaggaggccatgtttcgatctgcggatatgacctatatcgagttgtacattccgctagagatagcgcgcgaggttgtttgtgtattaggtaacctggggagcgtaatgttaaaggacatgaacaaagacctaagcacgttccagcgcgggtacgttaaccaggtgcggaggttcgatgaggtcgagcggcaggtggggtacatggagggtgtggtacggaggcacaagaacgagacatggcggtacctgtatcgacatctacagcgggaagagcagcaggagtatcccggccgggagcaccccacgcttgcgcagctgatcggttcaatgcacacacactcgattgattcggtggacgaggttgcggaggaaatcatgcagttcgaggggcgcgtgaggcagctcgaccagagtcttgtagcgatgcgcgagcgactgtcgaagcttgtgcatgagcgacgtgtgatgttcacttgcgaacactttctcgaagtgaaccccgggatcggggagcgattgcccacacgccccgcggggttcgaagctgatgagtttgaactcacgcgtgtcggggaggaggacgaggagacggcaagccagctgtcttttgatatttcggatgacgcagagacacagctgcccggggacatgcgcacgttactcgaaccggtgtaccggcatcagtacctacttacaggctcaattgagcgtgctaaggtagaggcgctcaacaagatcttgtggcgccttctccgtgggaatgttttcttccagaatttccctgtctcagtgtcaccggtggaagaagatgataccgatctcgagactgactgctttatcgtctttacgcatggcgaagtcttgctaagcaaggcgaagaaagtgatagaatccctaaatggcacaatatatccgtttatgcaagacggggcgacagtgcaggagttgaacgacaagatagcggaccttaagcagatatgttctacgacagaacagacactacatacggaactttttcttgttgccaaccaattgcccatgtggaatgccattattaagcgtgaaaagtacatctactctgccttgaatttatttaggcaggagtcacagggcctcgttgcagagggatggcttcccacgtacgatctaccaggagttcaggcggcactaaaggactatggggagagcgtaggatccgcaaattcggccgttttgaatgtaatttcgacaacgaggacaccgccaactttccacaggactaacaagtttacgcaggcattccagtccattgttgatgcctatggtattgcaacatataaagaagtgaaccccgggttggcaactatcgtcacatttccctttatgtttgccgttatgtttggcgatgccggacacggtgcattgatgctcatagccgcgctctatttggtgttaaatgaaaagaagttgggagcaatgaaacgaggtgagattttcgacatggcatacactggaagatatgttattctactgatgggaatcttttctatctataccggtataatgtacaatgatattttttccaagtccatgcatttgttttccactggctggaaatggccttcaaactttcaagagggcgagatgattgaggctcaaaaagttggtgtttacccatttggattggactatgcctggcacggttcggacaatagtttgttattcaccaattcatataaaatgaagttatcaatccttctaggctttatccacatgtcgtattcatatattttctcatatctcaactaccactataaaggttcgaggatcgatattgtcggcaacttcattcctggattaatatttatgcaatcaatatttgggtacctatcgtgggcaattatttacaagtggtcaaaagattggattaaagatgagctgcctgcgcctggtttattaaacatgttgattaacatgtttttatctcccggagttgtcgatgaaaaactatatacaggccagagctttcttcaagttatccttttgctagctgctctagtttgtgttccgtggctactactttacaaaccgttgatgttgaagcgccagaacgatatagctctcagcaaaggattcagaagcctcagagaccaacgggtgcatgagattcttctggaagcacaggaaaacgcaggcgaagatatgctggttgcagattatgaaaatgaagatgaatcgtcggaggagttcaactttggtgacgttatgatacatcaggttatccacaccattgaattttgtttgaattgtatatctcatactgcatcgtatctcagattgtgggccttatctcttgcgcatgctcaactctcgactgtgttgtggtctatgaccattcagaattcgttctccgactccaaccctggttcatttttctctgtcaccaaggtggtggttttgtttgccatgtggtttgtgttgactgtttgcattttagtcttaatggaaggaacgtcggctatgttgcactcgttaagattgcattgggtggaagcgatgtccaaattcttcgagggcgaaggctatgcttacgaaccattttccttcaaggctatcaacagcgatgatgaatagtatcgtatatattaaaactagacagatggaggtagtgtcacatgtgtcaggtatagattgcccgatgaataagcatccttgaaatttagaaaaatcataggcctttagaatcgacatacaacgtctaaatatatttactattcacttcatagttcatcgtgatcgatgtccgaagtggaactatctgcgctagaggtggatgcatctgctggttgttgcgtagaggtcgcttttgcttcatctgcgaagtcagtagtagatgaagaggctgcttctttgcttttctcttctttcctctttgccgctcttagttttcttgtgtataatgagtttagctcacgcaagcggtatgcatgtgctctttcgcggaatttttgcatttcctcgatggaatttagatgaccttcggatttcatgaacaattttactaactgttcgcggtcaattgtatcaaatgcatctggagttaatatgggctcgagttcgctcagtatcttaccgagtgcggccaattctgactcagcaaaggttgaattgtactgtaaattctttggaaccttcagtttagtatattctctagtgacatttaggccaagagagttaaaattatcctcaagttcattagcttgtgccactgcagtatcatcaaatgcgttaaaggtagaaatggcttcagaaacatttttgcgtagaagctcaaatactgggagatccaaaaccttagcgtctatggaaaggtaatggctaagcaacttatacagactcatggtttcctggaatctgtggtggatatcgcca

SEQ ID NO: 22 - protein, 877 aa, Ashbya gossypii
  misc_feature: Oligo 31
MFLSMRAEEAMFRSADMTYIELYIPLEIAREVVCVLGNLGSVMLKDMNKDLSTFQRGYVNQVRRFDEVERQVGYMEGVVRRHKNETWRYLYRHLQREEQQEYPGREHPTLAQLIGSMHTHSIDSVDEVAEEIMQFEGRVRQLDQSLVAMRERLSKLVHERRVMFTCEHFLEVNPGIGERLPTRPAGFEADEFELTRVGEEDEETASQLSFDISDDAETQLPGDMRTLLEPVYRHQYLLTGSIERAKVEALNKILWRLLRGNVFFQNFPVSVSPVEEDDTDLETDCFIVFTHGEVLLSKAKKVIESLNGTIYPFMQDGATVQELNDKIADLKQICSTTEQTLHTELFLVANQLPMWNAIIKREKYIYSALNLFRQESQGLVAEGWLPTYDLPGVQAALKDYGESVGSANSAVLNVISTTRTPPTFHRTNKFTQAFQSIVDAYGIATYKEVNPGLATIVTFPFMFAVMFGDAGHGALMLIAALYLVLNEKKLGAMKRGEIFDMAYTGRYVILLMGIFSIYTGIMYNDIFSKSMHLFSTGWKWPSNFQEGEMIEAQKVGVYPFGLDYAWHGSDNSLLFTNSYKMKLSILLGFIHMSYSYIFSYLNYHYKGSRIDIVGNFIPGLIFMQSIFGYLSWAIIYKWSKDWIKDELPAPGLLNMLINMFLSPGVVDEKLYTGQSFLQVILLLAALVCVPWLLLYKPLMLKRQNDIALSKGFRSLRDQRVHEILLEAQENAGEDMLVADYENEDESSEEFNFGDVMIHQVIHTIEFCLNCISHTASYLRLWALSLAHAQLSTVLWSMTIQNSFSDSNPGSFFSVTKVVVLFAMWFVLTVCILVLMEGTSAMLHSLRLHWVEAMSKFFEGEGYAYEPFSFKAINSDDE

SEQ ID NO: 23 - DNA, 872 bp, Ashbya gossypii
  misc_feature: Oligo 4
gatctcatggaggattgattatataactatttcttgctgagaggaagttatggctataatcgccgtgtacacatgtatttccaagacgctcactttttcttcatagagggtgctccatataacctgatagagcttaagcgggtctttgaaaccaaagataaacttcttaaagtttattccatattactcgtaaaatggaactctaaagttttacagcctcaagggtgaggcagtgacgacttttttcatccattccggcaccttgagaatatttgatgttaagtgctctgttgttagtagatagctttcatggctatctcgaatgtttgactctgtattggctccgcctgagtcaagcttttgaagctaagaaaggaccaacttgtaaaacttcagacaaattttatctaggaacatggcctgattcagctcgaggtatcctctttcaactcaaatcacattaatgcactaaaactgagtgtaagcaagacctgcaaataataaaatttgggcacattttcattaaatttctcccatataagatagtatcttctcatgtaaagcatattaatgaaacccacacaaagaccagaagtattatactgaaaagtataggaagaaacagcaataatcatcccattattaaataattctactaaaccggtcaggataaggtatatttcagacatgagactgagctttttccatagtccgaagaacaatccttcacacgccacaattaaccagcaccatttcattataggatagaaatgttcagaaattctctttggatcaatagaatctcacatgaaagaagtattgacgtattttctgattcctgggcatgtaaacttaaaccgctgttaaggatc

SEQ ID NO: 24 - protein, 114 aa, Ashbya gossypii
  misc_feature: Oligo 4
PQRFKFTCPGIRKYVNTSFMDSIDPKRISEHFYPIMKWCWLIVACEGLFFGLWKKLSLMSEIYLILTGLVELFNNGMIIAVSSYTFQYNTSGLCVGFINMLYMRRYYLIWEKFN

SEQ ID NO: 25 - DNA, 2093 bp, Ashbya gossypii
  misc_feature: Oligo 4
ccatgaatgagcgataatatatataacaagagtggtgtagcatggcatgagcctgcttgcggatggataaaggaggagtcttcaggataggaataatttcccaagaatctattcatgaatggtatccacaataggtctttaatgtgagttacgtagagtctcttatgagatttttacagaaatttgattttaaaaatggtcatttagcatgtttagtctatgtcgccaggggaatggtgtttaaaaattttctcatttgttgtgaattcaagatatgctatgatacacaaataaacgatatacggactctaccacatgcaaattatagactaaaaagatacggttgataaaagcatttttaaggagcaagattaaccgtccagttgcgtaatattatacttaatttggtttatagaggtgtttttactcgtcttcatgatgtttatcggtattggataaacatgatggcaccactctctatcatatcgctcgcagaatagccgatataatcttttcagtagcgaaaaaatattttgcgttatcttaaataaatttaagcttagttaccattttttttatttagagaactgtctactgtgatctcatggaggattgattatataactatttcttgctgagaggaagttatggctataatcgccgtgtacacatgtatttccaagacgctcactttttcttcatagagggtgctccatataacctgatagagcttaagcgggtctttgaaaccaaagataaacttcttaaagtttattccatattactcgtaaaatggaactctaaagttttacagcctcaagggtgaggcagtgacgacttttttcatccattccggcaccttgagaatatttgatgttaagtgctctgttgttagtagatagctttcatggctatctcgaatgtttgactctgtattggctccgcctgagtcaagcttttgaagctaagaaaggaccaacttgtaaaacttcagacaaattttatctaggaacatggcctgattcagctcgaggtatcctctttcaactcaaatcacattaatgcactaaaactgagtgtaagcaagacctgcaaataataaaatttgggcacattttcattaaatttctcccatataagatagtatcttctcatgtaaagcatattaatgaaacccacacaaagaccagaagtattatactgaaaagtataggaagaaacagcaataatcatcccattattaaataattctactaaaccggtcaggataaggtatatttcagacatgagactgagctttttccatagtccgaagaacaatccttcacacgccacaattaaccagcaccatttcattataggatagaaatgttcagaaattctctttggatcaatagaatctcacatgaaagaagtattgacgtattttctgattcctgggcatgtaaacttaaaccgctgttaaggatcgcaatagttctcaatattatgaagttgccaattaataacgccaagattcacaaagatctgtataagaatcattaaaagctgaccccgaaatattggaacgggtggtatctttgcgtaatgtgccaacttaatatcgccaagatagttatcagtttggatatcaatattatacgcgaatgacttgataaaagttagcgcataatgatttctcggcacttttcaaaccacttattacctgcaacagcagcattgttgctatagtaatactatctgtgacttgtagcaacgcgacggaaagtgatagatcggagttctaatctatggaaaaccatattgctcagatcaattcgacttccaaggctatactaccagagatattgcaaaggttgtgaagaacggtccctacagtcactcctgctgccattggttcttaagagataccatatcttaaataccccttgtcgggcaatgatgaggattagcaacattcattgatagttggtatccttttcttagtaccaatgcggtgtgcaggtgtaatggtttacaggcggaacaatggaggatatgcagtttctggacaaggataatcaaggaatgaaaccattaggagtgggccaata

SEQ ID NO: 26 - DNA, 2093 bp, Ashbya gossypii
  CDS (738)..(1037)
tattggcccactcctaatggtttcattccttgattatccttgtccagaaactgcatatcctccattgttccgcctgtaaaccattacacctgcacaccgcattggtactaagaaaaggataccaactatcaatgaatgttgctaatcctcatcattgcccgacaaggggtatttaagatatggtatctcttaagaaccaatggcagcaggagtgactgtagggaccgttcttcacaacctttgcaatatctctggtagtatagccttggaagtcgaattgatctgagcaatatggttttccatagattagaactccgatctatcactttccgtcgcgttgctacaagtcacagatagtattactatagcaacaatgctgctgttgcaggtaataagtggtttgaaaagtgccgagaaatcattatgcgctaacttttatcaagtcattcgcgtataatattgatatccaaactgataactatcttggcgatattaagttggcacattacgcaaagataccacccgttccaatatttcggggtcagcttttaatgattcttatacagatctttgtgaatcttggcgttattaattggcaacttcataatattgagaactattgcgatccttaacagcggtttaagtttacatgcccaggaatcagaaaatacgtcaatacttctttcatgtgagattctattgatccaaagagaatttctgaacatttctatcctataatgaaatggtgctggttaattgtggcgtgtgaaggattgttcttcggactatggaaaaagctcagtctcatgtctgaaatataccttatcctgaccggtttagtagaattatttaataatgggatgattattgctgtttcttcctatacttttcagtataatacttctggtctttgtgtgggtttcattaatatgctttacatgagaagatactatcttatatgggagaaatttaatgaaaatgtgcccaaattttattatttgcaggtcttgcttacactcagttttagtgcattaatgtgatttgagttgaaagaggatacctcgagctgaatcaggccatgttcctagataaaatttgtctgaagttttacaagttggtcctttcttagcttcaaaagcttgactcaggcggagccaatacagagtcaaacattcgagatagccatgaaagctatctactaacaacagagcacttaacatcaaatattctcaaggtgccggaatggatgaaaaaagtcgtcactgcctcacccttgaggctgtaaaactttagagttccattttacgagtaatatggaataaactttaagaagtttatctttggtttcaaagacccgcttaagctctatcaggttatatggagcaccctctatgaagaaaaagtgagcgtcttggaaatacatgtgtacacggcgattatagccataacttcctctcagcaagaaatagttatataatcaatcctccatgagatcacagtagacagttctctaaataaaaaaaatggtaactaagcttaaatttatttaagataacgcaaaatattttttcgctactgaaaagattatatcggctattctgcgagcgatatgatagagagtggtgccatcatgtttatccaataccgataaacatcatgaagacgagtaaaaacacctctataaaccaaattaagtataatattacgcaactggacggttaatcttgctccttaaaaatgcttttatcaaccgtatctttttagtctataatttgcatgtggtagagtccgtatatcgtttatttgtgtatcatagcatatcttgaattcacaacaaatgagaaaatttttaaacaccattcccctggcgacatagactaaacatgctaaatgaccatttttaaaatcaaatttctgtaaaaatctcataagagactctacgtaactcacattaaagacctattgtggataccattcatgaatagattcttgggaaattattcctatcctgaagactcctcctttatccatccgcaagcaggctcatgccatgctacaccactcttgttatatatattatcgctcattcatgg

SEQ ID NO: 27 - protein, 100 aa, Ashbya gossypii
  misc_feature: Oligo 4
MKWCWLIVACEGLFFGLWKKLSLMSEIYLILTGLVELFNNGMIIAVSSYTFQYNTSGLCVGFINMLYMRRYYLIWEKFNENVPKFYYLQVLLTLSFSALM

SEQ ID NO: 28 - DNA, 853 bp, Ashbya gossypii
  misc_feature: Oligo 6
gatcatattccccttgtaggtctagccgcacgggtaaacggcaatgtatgtcgcagaacaagtccccacacctccggcagttctggattcctgtcctcggtgtgagctttcgcccgcatctcgagcattgtgacttccctggcacaagaggtttgtaatgtgacctgtttctgccggatgtctcccgactggacgccagacccaccgaagttggtgtagtacttgcaatgttcacsggaaccgctgtctgtggatagcgatcgttcatctttaaaattgtgttcaacatccagatgcatattcaactgttccaagttttcaatatttcgattgcatacggggcactcgaccgattccgaggcgtcagaggcagcagcattactgtactctgtaggtatttgttcatctgcctccggggttggcattactgggccacctagttggaactgacatttatgatataacgcacagcactaatgtgtgaaacgcaaaagttcaccacactgtcaaaaaccaccacacagtcaactcgagatagacacaataatgatggaagagctgtttatgctaccagttattggtatcacggcgttgttggtgtataaatacgcttacgatatcctttggtacaagttacagaatctatttgaactgattccttcatcaagctcatctcacccctactcttcggctatcgctagcattaataagtctcagaatgggtttctgtacaagctatacacggaatattcagtttcttctaacaatgtgctgcgagtgattcggactgctagtctccgggactgttggctgctgtgcgtctgtggctgtggagatagtgctatggcagatc

SEQ ID NO: 29 - protein, 38 aa, Ashbya gossypii
  misc_feature: Oligo 6
ESVECPVCNRNIENLEQLNMHLDVEHNFKDERSLSTDS

SEQ ID NO: 30 - protein, 55 aa, Ashbya gossypii
  misc_feature: Oligo 6
RSHYKPLVPGKSQCSRCGRKLTPRTGIQNCRRCGDLFCDIHCRLPVRLDLQGEYD

SEQ ID NO: 31 - DNA, 2389 bp, Ashbya gossypii
  CDS (428)..(1993)
gatctgccatagcactatctccacagccacagacgcacagcagccaacagtcccggagactagcgtccgaatcactcgcagcacattgttagaagaaactgaatattccgtgtatagcttgtacagaaacccattctgagacttattaatgctagcgatagccgaagagtaggggtgagatgagcttgatgaaggaatcagttcaaatagattctgtaacttgtaccaaaggatatcgtaagcgtatttatacaccaacaacgccgtgataccaataactggtagcataaacagctcttccatcattattgtgtctatctcgagttgactgtgtggtggtttttgacagtgtggtgaacttttgcgtttcacacattagtgctgtgcgttatatcataaatgtcagttccaactaggtggcccagtaatgccaaccccggaggcagatgaacaaatacctacagagtacagtaatgctgctgcctctgacgcctcggaatcggtcgagtgccccgtatgcaatcgaaatattgaaaacttggaacagttgaatatgcatctggatgttgaacacaattttaaagatgaacgatcgctatccacagacagcggttccgtgaacattgcaagtactacaccaacttcggtgggtctggcgtccagtcgggagacatccggcagaaacaggtcacattacaaacctcttgtgccagggaagtcacaatgctcgagatgcgggcgaaagctcacaccgaggacaggaatccagaactgccggaggtgtggggacttgttctgcgacatacattgccgtttacccgtgcggctagacctacaaggggaatatgatccgagaaacggcgactggtgcaagtgctgccatgcctgcatggcggggcgccctggatacaacaaactgggcttatcggtggaccggaccgacgagtttatacgccatcgtacctctaaaaacgaggataaacagcttcgcattctgcagcttgagaatcggcttgtgcgtcttgttgacggcatcgctactattgttcgcgcgcacaaccagtctttgttttatggaagtggaatgtatagggaaatcactgctctacagaagtccgttacgccatggaaagaaaattcacaggcatcaagctgctatctctgttctcggccgttcaatcttcttcttcgcaagcaccattgcaagttgtgtggtctgatagtctgcgaaaacaactttaccaactgttccaaggagttcccgatcgcgcagctggtgagtgctgccacagacctaccattcaggagcaatccccaggaattagccgcattgccggttcgactacgcgtctgcgtggtttgcataagatccgtgtttctgcgcgcaaggttgcaggacaatctcgcaaacgatgcttctcagctattttccaagtacactgagttgcagcgtgtatcaagagccatcttgcgtataatgcctcgtttcgagcaactgctcggggacctcaacgcacccgacgcgagcccgaatcgaagcgagctagacgagctggcgcacctgcgaaggaaactcttggagactttcaagctgtacgacaccatagccaagcagatatttgccatcgacccggccaataccgcagaattgaagatccagcaggctataaaggccaaatcgatgtccttcatacaggataaaatgctaccgctaaagaatatccccggcctgctgaagccgaaggatgccgaacctgagatcaattacactactagcaatctacttttcaacaatctgactgtccgcgaggttaaactctaccgtgaacagctgatggtactcaaagagcagaggtttatagtggagggcatgctcgagaacgccaagaaacagcggcgttttgaagaggttaatacgttaaaggaaaataccaaagagctagacaatcagatagcccagctcgaagaaaccctaggcgaccagggttttgtttagtatctagcatggagttttttgcttaactataattactgtgtagatgccgcagatagcatgtcgtagcataattgcgaattttcaccaacatgaaaaagtgtatgtgtataaggcatccagtgaactcctaacatgctgatgaggttttaagtaaagatatcactagcaatgaacgtaagtgcagtttttgagctttatgtcctctgtagaacataatattaacgacagggggataggatgaaagaagacagcagttatttgagctgaacagtgaagcctggtctggaattgatgcgttcccgaataaaaccagcaagcttgactcaagcatcaagagaaacacagggtttatcaaaaagctgaaacagggtatcacgaaagactcgaaagatc

SEQ ID NO: 32 - protein, 522 aa, Ashbya gossypii
  misc_feature: Oligo 6
MPTPEADEQIPTEYSNAAASDASESVECPVCNRNIENLEQLNMHLDVEHNFKDERSLSTDSGSVNIASTTPTSVGLASSRETSGRNRSHYKPLVPGKSQCSRCGRKLTPRTGIQNCRRCGDLFCDIHCRLPVRLDLQGEYDPRNGDWCKCCHACMAGRPGYNKLGLSVDRTDEFIRHRTSKNEDKQLRILQLENRLVRLVDGIATIVRAHNQSLFYGSGMYREITALQKSVTPWKENSQASSCYLCSRPFNLLLRKHHCKLCGLIVCENNFTNCSKEFPIAQLVSAATDLPFRSNPQELAALPVRLRVCVVCIRSVFLRARLQDNLANDASQLFSKYTELQRVSRAILRIMPRFEQLLGDLNAPDASPNRSELDELAHLRRKLLETFKLYDTIAKQIFAIDPANTAELKIQQAIKAKSMSFIQDKMLPLKNIPGLLKPKDAEPEINYTTSNLLFNNLTVREVKLYREQLMVLKEQRFIVEGMLENAKKQRRFEEVNTLKENTKELDNQIAQLEETLGDQGFV

SEQ ID NO: 33 - DNA, 975 bp, Ashbya gossypii
  misc_feature: Oligo 146
gatccggacgacagggctgcggcacaggagtcgtaccgtaggggcggggcagtccgaggccagcacgaatgggttccggccgttcacggacgagggcatgttcttccgaggggccgggggggctccggaggacctgtttgactttttctttcggggcgggggccccggcggcccgttcgggatggctgatccctatgattcgtttgggccctttgggggcgctacgacgtttacgtttggcgggcccgcgggcttcaaggtgtacagcggagggtctggggggcagttccgccgcggaccgtttggcatggcacaggctgccaccgaagcgcaacgccaacgtgcgaacggccagccggaggcgcaagatccgctacaacacgtagtgttcgttctcctgatagtgcttctcttcctgctactgccaagtctgggcttctgacttggagcagtatccgttgtaccatacagggccggcgccgacctgaccaaggcttcccatggctggccttccatgatggcatttcgtaaatacctatttttatagtcaacaagtataaataaataataatatacaaataatttacaaatctgcagcacgtcgcgtgacgctgactgcgacataaaaatgttcaagatagtttgattagaatgcacaaacaaacattgcaacacttacataaaccagatgaagctacagccgctattccaggttttgcattctgcgttctattatttcgtcaatatcctcggctgctggcttaagctttagcttcctgcctaatttggcggcaaatccatcgtttcccgagccgccagaattgtcaggtactagattttttctcttgataagtttgccactcggtaactgctcatattcctctgtgttatatgtggcactgctgtgtatagtcactttgctaggcgttccaggaggtaactcggtcgcaccgggtagtgtattcttcctggatc

SEQ ID NO: 34 - protein, 66 aa, Ashbya gossypii
  misc_feature: Oligo 146
SSATYNTEEYEQLPSGKLIKRKNLVPDNSGGSGNDGFAAKLGRKLKLKPAAEDIDEIIERRMQNLE

SEQ ID NO: 35 - DNA, 2509 bp, Ashbya gossypii
  CDS (882)..(1208)
ctgcagtttctgaaggtgctggtggcagatccctacggtttgaacaagatccgcaccgcgttcgagaagggctacatcacgccgaatgacacatggttccagggtggcacgacgacatacgacaacattgcctatacgctgcgcctgatgatcgactgcggcattgttggcatgtcgtggatcacactaccaaagggcaagtatgccatgatcccaaagaacaaaaaaatatctacgtgccagctggaagtgtcaataaactataaggacctgatttcgcggccggcagacagtgactggtcgcacagtgctccattgcgaatcctctctttcgacatagaatgtgctggccgcgtgggcgtgtttccagaaccggaggttgatcctgtcattcagatagcgaatgttgtcagtattgcgggagagtcaaaaccatttattcgcaatgtcttcacagttgacacctgtgcgcccattactggctcgcaaatttttgagcatgagacagaagaagcgatgcttaaacactggcgtgacttccttgttgaggttgaccccgacgttatcattggttacaatacgacaaattttgatcttccttacctgatcaacagagctgcagccttgaacgtgtcttcatttccctactttggccgtttggttaactccaagcaggtcatcaaagaaactgtattttcatccaaggctcatggaacgcgggagtcgaagtcgatcaatatcgagggccgccttcagttggatatttttcagtttgtaagacgagaatatcagttgagatcgtacactttgaattcggtgcgtttaatgagttgaagcaaggatggacatggcaacgtgatcctccaacaattaagcgcttgatgaacaaggcgattggaaataaaagcgatacttttgatggcgttatcaaccatacgcgatccaggaagaatacactacccggtgcgaccgagttacctcctggaacgcctagcaaagtgactatacacagcagtgccacatataacacagaggaatatgagcagttaccgagtggcaaacttatcaagagaaaaaatctagtacctgacaattctggcggctcgggaaacgatggatttgccgccaaattaggcaggaagctaaagcttaagccagcagccgaggatattgacgaaataatagaacgcagaatgcaaaacctggaatagcggctgtagcttcatctggtttatgtaagtgttgcaatgtttgtttgtgcattctaatcaaactatcttgaacatttttatgtcgcagtcagcgtcacgcgacgtgctgcagatttgtaaattatttgtatattattatttatttatacttgttgactataaaaataggtatttacgaaatgccatcatggaaggccagccatgggaagccttggtcaggtcggcgccggccctgtatggtacaacggatactgctccaagtcagaagcccagacttggcagtagcaggaagagaagcactatcaggagaacgaacactacgtgttgtagcggatcttgcgcctccggctggccgttcgcacgttggcgttgcgcttcggtggcagcctgtgccatgccaaacggtccgcggcggaactgccccccagacccaccgctgtacaccttgaagcccgcgggcccgccaaacgtaaacgtcgtagcgcccccaaagggcccaaacgaatcatagggatcagccatcccgaacgggccgccggggcccccgccccgaaagaaaaagtcaaacaggtcctccggagcccccccggcccctcggaagaacatgccctcgtccgtgaacggccggaacccattcgtgctggcctcggactgccccgcccctacggtacgactcctgtgccgcagccctgtcgtccggatcgtagcccagctggtcgtacacgcggcgcttcttctcgtcagacaaaacctcgaacgcacgatttaccttcttgaacgcttccgccgccctcgggtgccgatttttgtccggatgtagtttgattgccatcttgcggtacgccttctttatgtccccatcgctcgccttctcgtccacctgtagcagctcatagaacgaatgcttgtccttatccacaatcagcagcgtcagcttttcttgctcttctgtgtactctttctccgccatcttgatagctatcttagctctccgccaatctgccagaccttgttcttcttgccaccgagctacaattcctcgagcctctcaaccaaactaccttataacagctccttcagatcacacgctgctggaatcgatgactggtccccggtctgtcactattcatgcacaaagcaactatagaacactcctcttttcttaaatgtaaaagttgaagccaccgtgcagcgccgtttacgggaagttccccaaggtagcacaacagaaggtaaagatgaaacgactacgcacccgcataccggcctagggccaacggcgacattcggcgtacagc

SEQ ID NO: 36 - protein, 109 aa, Ashbya gossypii
  misc_feature: Oligo 146
MNKAIGNKSDTFDGVINHTRSRKNTLPGATELPPGTPSKVTIHSSATYNTEEYEQLPSGKLIKRKNLVPDNSGGSGNDGFAAKLGRKLKLKPAAEDIDEIIERRMQNLE

SEQ ID NO: 37 - DNA, 1031 bp, Ashbya gossypii
  misc_feature: Oligo 56
gatccactaataattggacgatacgcgcgtacccttcagtcgctgccgcaaagattggggtccagccaaaaagcttctcaccaagttccatatttgcaccctgtttaactagatattcggccgcctcatagatgtctaacttgcaagcaacataaagagcagtctcgagattttccggctcctgatagtcaatatcaaagcccttagcagacaataaagaatttaacaaagacggcgagtttagtcttgtcgcgaggtgtagcaaacgagggctgttaagagatttctctgggttcataaacgacaatagcgtgctgacagtaagagggtgtgtcccgataacagccaaatgcaatggtgtgaggttctccgaatctccccacacgtcaatatcatcgattgcaacgtctgcattccaagcatcccactcactcagagcytgcagaattatcctggtamcctcacatagaccatattgggcagagtaatgtagcggcgttcgcttgtagttatctctttgtaacagcgacggtcttaaatgggctggcaactgttgtaatatgtgtgacaggggagctggagagtcgtcagagttaacgccatccggaccaaaagcaccaacaagtctagtattgggttccgggggtatcgctgcctctagatcgagagagtcgctcagtagcgagtttatgttatcacgctcttcgagactcttggtccgctttttgcccagtgcaattacatgatgatggaagaagtttctgccatttatatcggatggatctccgagcgtaggtataataccaaggatttcatccacacagctgaaggaccgcgataaagcggacttattcaatagcgtcaccagcgtccgcgtcggtatcaggacaacagaactggtacatagaaatgagctcgtttatcaagcccgccccgtcatccttggtaactagctgggacgccacatcaattggcgacgacgagccttggagcagacgacggtcttcgccgcgtagtttatcctgtagatc

SEQ ID NO: 38 - protein, 17 aa, Ashbya gossypii
  misc_feature: Oligo 56
VTKDDGAGLINELISMY

SEQ ID NO: 39 - protein, 298 aa, Ashbya gossypii
  misc_feature: Oligo 56
SSVVLIPTRTLVTLLNKSALSRSFSCVDEILGIIPTLGDPSDINGRNFFHHHVIALGKKRTKSLEERDNINSLLSDSLDLEAAIPPEPNTRLVGAFGPDGVNSDDSPAPLSHILQQLPAHLRPSLLQRDNYKRTPLHYSAQYGLCEXTRIILQALSEWDAWNADVAIDDIDVWGDSENLTPLHLAVIGTHPLTVSTLLSFMNPEKSLNSPRLLHLATRLNSPSLLNSLLSAKGFDIDYQEPENLETALYVACKLDIYEAAEYLVKQGANMELGEKLFGWTPIFAAATEGYARIVQLLV

SEQ ID NO: 40 - DNA, 4925 bp, Ashbya gossypii
  CDS (426)..(4388)
ctcaaggtttggctgggtctggaactacgccagctcatgacgctatccgtgcgtatcatcaatgacctcgttaaaacggatcagctatgtcctagttactgaaggccaaaaagaaatatataaaagtaacgctggtgcctttccaccttatccacgtgcggaagctcgggcgttaaggttacgagctacgcttgcagattgtgtttgaggaaggccagggtgctcgtgcctagcattcattccgcgcacatgtgtacatatatactagctttataggccacacaacaaacagatatgcgcacggctggagatatctcggcatacaccgcgtggcacagtgtttttccttgacttctgcatattagaggtttgtataggggatactagcttcagctggggagcacacagactaggctaggacaagtatgaagttcggcaagacatttcccaaccatcaggtgccggaatgggcacacaagtacgtgaattacaagggtctgaagaagcagatcaaggaaatcacgttggtgcaggatgcgctgttccgccaggagcaaggcgcagcttcgcaggacggacccgctcggcggcggggaagagagagcaaggagcagtatcttggccatccagaggtgaagaagctgcttgcagcatttttctttgccctggaccgggatatcgagaaggtggacggtttctacaacatgcagtttatggagtatgaccggcggctgaggaagcttctatcaagcgcgcagctggcagacatcacgtcggtacagcgcggcgctaccggctacctgcacgcgccacttccgcagtacatagcatacggggagcgcgaacgggatggattgccagagcgctatgtaccgccgcacgccactgacatgtcggaggacctagcggaggtgctgacgattctgctggagctgcggtcgcacttccgcaacctgaagtggtacggtgagctcaacaagcgggcattcacgaaaatcatgaagaaactggataagaaggttggcacaaaccagcagcactcctacttccaggcccgcattaagcccttagaatttgctgacgatacaccgatcgtcaaggcgctagctaccattaatgagatcctagatcgcatctcgccctgcgtgaaggatctacaggataaactacgcggcgaagaccgtcgtctgctccaaggctcgtcgtcgccaattgatgtggcgtcccagctagttaccaaggatgacggggcgggcttgataaacgagctcatttctatgtaccgttctgttgtcctgataccgacgcggacgctggtgacgctattgaataagtccgctttatcgcggtccttcagctgtgtggatgaaatccttggtattatacctacgctcggagatccatccgatataaatggcagaaacttcttccatcatcatgtaattgcactgggcaaaaagcggaccaagagtctcgaagagcgtgataacataaactcgctactgagcgactctctcgatctagaggcagcgatacccccggaacccaatactagacttgttggtgcttttggtccggatggcgttaactctgacgactctccagctcccctgtcacacatattacaacagttgccagcccatttaagaccgtcgctgttacaaagagataactacaagcgaacgccgctacattactctgcccaatatggtctatgtgaggttaccaggataattctgcaggctctgagtgagtgggatgcttggaatgcagacgttgcaatcgatgatattgacgtgtggggagattcggagaacctcacaccattgcatttggctgttatcgggacacaccctcttactgtcagcacgctattgtcgtttatgaacccagagaaatctcttaacagccctcgtttgctacacctcgcgacaagactaaactcgccgtctttgttaaattctttattgtctgctaagggctttgatattgactatcaggagccggaaaatctcgagactgctctttatgttgcttgcaagttagacatctatgaggcggccgaatatctagttaaacagggtgcaaatatggaacttggtgagaagctttttggctggaccccaatctttgcggcagcgactgaagggtacgcgcgtatcgtccaattattagtggatcatggggccaaatatgatctttttgatgagagcggttggacaccaatggagcacgcggctctgcggggacatttggatatctctcaactcattcgcataacagataataaagcgatcactcggccgaagttcgccaccgactggaacaagtctaccagaccaacagaaaccacgaatggtttgttatccgcattaacaccatcagaatctgggtctacaaccacgggctccgaaaacaagagctcttctttgacacctagcaccagcaatgaaatgtatgctctgccagcacgctcttcaacgtcaatagacaagatatctgaaccaaacaaaggaaaccacaggaaggttttgaagtcccagctaagccacggtaaggtacaaactattaaggacacacaattgccgcagcagccgattaagtctttcggtcatagcttcctgcagaaagacgagtctgttatccttttgactttgggcactaatgataatcgcagtaccatacctgctgtttctctgaataaggttcctgttgctaaggcttcctcaacggagctagatacagcgttatctttgttggttacatgcatggataatttggatgcagaacctgttatgctggaccttccattgcatgagaacttggattcggtcacttttaaagtcccatacaagaaagattcctcttatactatattttttgatatcgttcccacctatggctattcaatggcaaacatgaaccgcgaaaattcctctggtatgcattcgaatgttggtaatagtactggccccgcgtacctagacgcacaagtgggccagtgcggctctcgtttacactatgaccagcttgggagagatacaccaaacacttacgatcagcgttcgcggcaccaagcatcccaacaaaaggaacaaattgccacgaagaagcaaagtaagatattagggagggcagttgctttattggactccgcgccaacttcggtgggccccaacaggcgctctattgcggaggctatcactatacctatcattgggagcgacacacttgaagtcctcgggattattcgatttgacttcctcgtagttactccgtttgtccacaagaatttatcagttggacctgctgaaacgtattggaaatcgctggtttcgacccgggtgatcggacacagaggcttgggcaaaaacatgaacacgaacaagtcgttgcagcttggcgagaatacggtggagtcctttatcgcagctgcctcattgggtgcctcgtatgtcgaattcgatgtccagttgacaaaggataacattcccgttgtgtaccacgattttcttgttgcagaatcgggtgtcgatattcccatgcatgagctcactttggaacagtttctcgatctgaacggggagcgtcaaaggcatcaggacgcgagagaggcccacagaaaccacaggagcccgaacggcagacgcttgtccatggatgacagctctgctgagctaattaagcggtccctgatgatgcggggcgatgaagaccgcacagctagagaccttaacacaatatacggcgaccgtatgcggctgaccagaacgttcaagaagaatgccttcaaagccaactccaggggtcatgccattgcatctagttttgttacgctaaaagagctgttcaagaagatcccccagaatgttggcttcaacatcgagtgcaagtatccaatggtagacgaggccgaggaggaagacatcggcccgatcgccgtggaaatgaaccattggatcgataccgtgctggaggttgtctacgacaacgtcgagggccgtgacgtcatcttttcgtcgtttcagccagacgtgtgcctcatgctctccctaaagcagccctccttcccgatcctgttcctaacggaaggtgggaccgcgaagcgctgcgacatccgcgcggcgtcgctgcagaatgccatccgcttcgcgcaccgctggaacctgctgggcatcgtctcggccgcggcgccaatcgtcatcgcgccccgcctggcccagatcgtcaagtccagtggccttgtgtgcgtgacgtacggcgtcgagaacaacgaccccgagatcgcccgcgtcgagatggacgccggcgtcgacgcggtcatcgtcgacagcgtgctcgcggtccgcaagggcctcacccgcgaggcacaggacgccgacacgctctaagaacaaatcgtgattcactaagcagaagatatatcgttatgtagagcctgccgggtcccaggctaatccacccattgccgtcacaaaatcgtaaccgagatgcgccgcccgtgcccccgcgcgccgccctgcagactcgcgcccgcgctctgcatcgtcagcttctccaagctgactaacttgtcggtcacgtgatcatccttgaacatttccccgatctggtgcttgcccgcgtcatccgtgaacacctgcaacgtcttcgtctgcgccgcctcgttgatgtggtggtactcgtccgggaagggctggatgtgctcgaccgtcagttcgttcaattggatgaacttcatcactgtgaggttcgacacctcgactggaatattcgaaactatcacactgtgcgccatggggactctgtttgtactgccgcccttgttgcgccactgatgctgccaccgccacgtccagcttatatacccgcgtggaaagttgtcgaattgtgaaatagccgacgctatcggtaccatttacacc

SEQ ID NO: 41 - protein, 1321 aa, Ashbya gossypii
  misc_feature: Oligo 56
MKFGKTFPNHQVPEWAHKYVNYKGLKKQIKEITLVQDALFRQEQGAASQDGPARRRGRESKEQYLGHPEVKKLLAAFFFALDRDIEKVDGFYNMQFMEYDRRLRKLLSSAQLADITSVQRGATGYLHAPLPQYIAYGERERDGLPERYVPPHATDMSEDLAEVLTILLELRSHFRNLKWYGELNKRAFTKIMKKLDKKVGTNQQHSYFQARIKPLEFADDTPIVKALATINEILDRISPCVKDLQDKLRGEDRRLLQGSSSPIDVASQLVTKDDGAGLINELISMYRSVVLIPTRTLVTLLNKSALSRSFSCVDEILGIIPTLGDPSDINGRNFFHHHVIALGKKRTKSLEERDNINSLLSDSLDLEAAIPPEPNTRLVGAFGPDGVNSDDSPAPLSHILQQLPAHLRPSLLQRDNYKRTPLHYSAQYGLCEVTRIILQALSEWDAWNADVAIDDIDVWGDSENLTPLHLAVIGTHPLTVSTLLSFMNPEKSLNSPRLLHLATRLNSPSLLNSLLSAKGFDIDYQEPENLETALYVACKLDIYEAAEYLVKQGANMELGEKLFGWTPIFAAATEGYARIVQLLVDHGAKYDLFDESGWTPMEHAALRGHLDISQLIRITDNKAITRPKFATDWNKSTRPTETTNGLLSALTPSESGSTTTGSENKSSSLTPSTSNEMYALPARSSTSIDKISEPNKGNHRKVLKSQLSHGKVQTIKDTQLPQQPIKSFGHSFLQKDESVILLTLGTNDNRSTIPAVSLNKVPVAKASSTELDTALSLLVTCMDNLDAEPVMLDLPLHENLDSVTFKVPYKKDSSYTIFFDIVPTYGYSMANMNRENSSGMHSNVGNSTGPAYLDAQVGQCGSRLHYDQLGRDTPNTYDQRSRHQASQQKEQIATKKQSKILGRAVALLDSAPTSVGPNRRSIAEAITIPIIGSDTLEVLGIIRFDFLVVTPFVHKNLSVGPAETYWKSLVSTRVIGHRGLGKNMNTNKSLQLGENTVESFIAAASLGASYVEFDVQLTKDNIPVVYHDFLVAESGVDIPMHELTLEQFLDLNGERQRHQDAREAHRNHRSPNGRRLSMDDSSAELIKRSLMMRGDEDRTARDLNTIYGDRMRLTRTFKKNAFKANSRGHAIASSFVTLKELFKKIPQNVGFNIECKYPMVDEAEEEDIGPIAVEMNHWIDTVLEVVYDNVEGRDVIFSSFQPDVCLMLSLKQPSFPILFLTEGGTAKRCDIRAASLQNAIRFAHRWNLLGIVSAAAPIVIAPRLAQIVKSSGLVCVTYGVENNDPEIARVEMDAGVDAVIVDSVLAVRKGLTREAQDADTL

SEQ ID NO: 42 - DNA, 1078 bp, Ashbya gossypii
  misc_feature: Oligo 167
gatccttcgagcgtagacgaagaaaaggtcaaacgcctgaaatcgacctttaccttttagctctcaactaatccggtcgttatcaatcgtgtaattctataccttaaatagactacctaaccagcacgcagtgtcccatacagggcttgcagtatggtgcctaggtctcattcgttgtattcttttgctctgcagctttcctttgggcccatgcagcgtaagccctgccgttcgcaatcaacggattatcatgcttgaaatagtatgggccagagagcgctgaatattgtctcttgtctaagttatacactctttccatttcttccttatagacgtcctcgataattttaacatctccctcatctatcttcgtgttgttggaggcggtgtataaatctgcatcgaatggccgaatgcaattgccgacatagaaganggtggatgtgtgcccctcacccaattcaaactcggatttggggttacgcamccgccctagatgttccataagccgccgtgctagctgcggatggtctggctgctgcttttgctgatgtggcctgtcactctctgtttcctctccaccntgagacgccatagatggcccaacagagtttcgtggcacgtcaatccctccgtctcatggctggttatgcgtcgcagtccggaatggggacagttatatacgcagcaaactgcagatcgacccacgcggcctgtgcccacgacacacattaccgtagtcgcaggcgcttatgctcacaaaagaaagccctgtatataaggtcctaattatatagagctcttacaacactttctgtttcatgaagaaggtgcgcaggtggttgacctgccagaagcatgtgccgccaagcacaaacaacgtcatgaccgcacaccatacaacccacgaattggcgctctcggacgcatctgcggaattgggcctccctctcactgcaccaacctgctgctctgttgctggatcgccttcaccttctcgccgagctgtagcacgcgcgaatgcaggtactgcagcttgtccttcttcttgggatccagcagtaggtccgatccgatc

SEQ ID NO: 43 - protein, 42 aa, Ashbya gossypii
  misc_feature: Oligo 167
DASESANSWVVWCAVMTLFVLGGTCFWQVNHLRTFFMKQKVL

SEQ ID NO: 44 - DNA, 2549 bp, Ashbya gossypii
  CDS (563)..(1216)
tatgccccgcggctgcgggcccgccagcacccggcacatccaaaccggcccatcaaacatgtactcctggttctgccgccccaccgccgcgaggtcccacatcttcactctccaatcccgcgagctgctccataggtaccgtccacattgcgaccacgccaccgactgcaccgctcgtacatggccgcccccatggtttcccagcaccgaaataggcttcttcgtatccatgtcgtagatcaccagagacccgttggagcatccaactgccaggtagtcaccgcctgggctgaactccagacaatcgcattgcagcgggatttcaaacgtatatgttagcgtctcagggtattccttcagtacactgaatggatcctgcaacaaccgatttgccatcgccccttccccgcctccaagcctccaacctgatgctttcctccaggctgtacctggaaagtaaaaaaaaattcgaacagcgaaccagatatggacaccgaattgaagatcttgttccccctggaatctcagtatatcgatcgatcaggcgctgaaggcttccaggatgaacttgaagacatttttacaggtactggtgctcggcatgttgccgttgcaggtcagtgggttctacttctatgtcaatggtggagaccgtaagtgcttccacagtgaattaatgaaagatgcggttttgaacctgaagtacaacgtgcaatcgtacgactcccagtctggaacgtaccgggacatgcgggacggtgaactaatgatgatgatcgacatagaggaggtgtttgacgacaaccaccgtgttatgcaccagaaattgccggcgtacggtacaactacgttcaacgcggttgactctggcgagcacaaggtttgcgttcagccgcagttccaggggtggatggcgcgcaccaagacaaagatcacgcttgactttgagatcggatcggacctactgctggatcccaagaagaaggacaagctgcagtacctgcattcgcgcgtgctacagctcggcgagaaggtgaaggcgatccgcaacgagcagcggttggtgcgtgagagggaggcccaattccgcgatgcgtccgagagcgccaattcgtgggttgtatggtgtgcggtcatgacgttgtttgtgcttggcggcacatgcttctggcaggtcaaccacctgcgcaccttcttcatgaaacagaaagtgttgtaagagctctatataattaggaccttatatacagggctttcttttgtgagcataagcgcctgcgactacggtaatgtgtgtcgtgggcacaggccgcgtgggtcgatctgcagtttgctgcgtatataactgtccccattccggactgcgacgcataaccagccatgagacggagggattgacgtgccacgaaactctgttgggccatctatggcgtctcagggtggagaggaaacagagagtgacaggccacatcagcaaaagcagcagccagaccatccgcagctagcacggcggcttatggaacatctagggcggttgcgtaaccccaaatccgagtttgaattgggtgaggggcacacatccaccctcttctatgtcggcaattgcattcggccattcgatgcagatttatacaccgcctccaacaacacgaagatagatgagggagatgttaaaattatcgaggacgtctataaggaagaaatggaaagagtgtataacttagacaagagacaatattcagcgctctctggcccatactatttcaagcatgataatccgttgattgcgaacggcagggcttacgctgcatgggcccaaaggaaagctgcagagcaaaagaatacaacgaatgagacctagcaccatacgcaagccctgtatgggacactgcgtgctggttaggtagtctatttaaggtatagaattacacgattgataacgaccggattagttgagagctaaaaggtaaaggtcgatttcaggcgtttgaccttttcttcgtctacgctcgaaggatcaatcccagatacaggtaaggttccgaagtcaaagtggaacggaaccgcttcggcagatgctgcagtgttctctgcaaatgaggagtcatccttgggtaagtcaggtttcgagaaggaaaatgcaaagggggtgcgggatgtcgtattgtgcttatcttcacttttatgaacttgggagactttctgaatgccattagaaaaattgaacgacccattcgactgcaatgagctccccgatatgttattctccatgggcccgttaagagtagctccgtgggtattagattcctctactttggccagaggttgaaacgaaaatgtgctggttgcatcaggtatagtgtgtggcttctcagacaaagtatccacattgtggtctttgggcttttggaaagaaaagttaaatttagagctctgtggaacaggctgccgaggcgacttttcaggcgacttttcaacagtcacctcaacgcgccagcgatgaaggcctggaaggtttaaacccggacactggttgtgtagcatgagga

SEQ ID NO: 45 - protein, 218 aa, Ashbya gossypii
  misc_feature: Oligo 167
MNLKTFLQVLVLGMLPLQVSGFYFYVNGGDRKCFHSELMKDAVLNLKYNVQSYDSQSGTYRDMRDGELMMMIDIEEVFDDNHRVMHQKLPAYGTTTFNAVDSGEHKVCVQPQFQGWMARTKTKITLDFEIGSDLLLDPKKKDKLQYLHSRVLQLGEKVKAIRNEQRLVREREAQFRDASESANSWVVWCAVMTLFVLGGTCFWQVNHLRTFFMKQKVL

(19) United States
(12) Patent Application Publication
(10) Pub. No.: US 2005/0148761 A1
(43) Pub. Date: Jul. 7, 2005

(54) Genetic products of ashbya gossypii, associated with transmembrane transport

(76) Inventors: Marvin Karos, Neustadt (DE); Henning Alhofer, Wachenheim (DE); Burkhard Kroger, Limburgerhof (DE); Jose L Revuelta Doval, Salamanca (ES)

Correspondence Address: James Remenick;Morrison & Foerster, 1650 Tysons Boulevard, Suite 300, McLean, VA, 22102, US

(21) Appl. No.: 10/485,986

(22) PCT Filed: Aug. 9, 2002
(86) PCT No.: PCT/EP02/08937

(30) Foreign Application Priority Data
Aug. 10, 2001 (DE) 10139454.3
Aug. 10, 2001 (DE) 1039455.1
Aug. 10, 2001 (DE) 10139457.8
Aug. 10, 2001 (DE) 10139458.6
Aug. 10, 2001 (DE) 10139459.4
Aug. 10, 2001 (DE) 10139460.8
Aug. 10, 2001 (DE) 10139461.6
Aug. 10, 2001 (DE) 10139462.4
Aug. 10, 2001 (DE) 10139463.2
Aug. 10, 2001 (DE) 10139464.0
Mar. 6, 2002 (DE) 10209819.0
Mar. 6, 2002 (DE) 10209816.6
Apr. 11, 2002 (DE) 10216033.3
Mar. 16, 2002 (DE) 10121911.7
Mar. 16, 2002 (DE) 10221928.1
Mar. 16, 2002 (DE) 10221909.5
Jun. 7, 2002 (DE) 10225390.0
Jun. 7, 2002 (DE) 10225392.7
Jun. 21, 2002 (DE) 10227796.6
Jul. 29, 2002 (DE) 10234455.8

(51) Int. Cl. C07H 21/04; C07K 14/195; C12N 15/74; C12N 1/21

(52) U.S. Cl. 530/350; 435/69.1; 435/320.1; 435/252.3; 536/23.7

(57) ABSTRACT

The invention relates to novel polynucleotides from Ashbya gossypii; to oligonucleotides hybridizing therewith; to expression cassettes and vectors which comprise these polynucleotides; to microorganisms transformed therewith; to polypeptides encoded by these polynucleotides; and to the use of the novel polypeptides and polynucleotides as targets for improving transmembrane transport and, in particular, improving vitamin B2 production in microorganisms of the genus Asbya.

The present invention relates to novel polynucleotides from Ashbya gossypii; to oligonucleotides hybridizing therewith; to expression cassettes and vectors which comprise these polynucleotides; to microorganisms transformed therewith; to polypeptides encoded by these polynucleotides; and to the use of the novel polypeptides and polynucleotides as targets for modulating transmembrane transport and, in particular, improving vitamin B2 production in microorganisms of the genus Ashbya.

Vitamin B2 (riboflavin, lactoflavin) is an alkali- and light-sensitive vitamin which shows a yellowish green fluorescence in solution. Vitamin B2 deficiency may lead to ectodermal damage, in particular cataract, keratitis, corneal vascularization, or to autonomic and urogenital disorders. Vitamin B2 is a precursor for the molecules FAD and FMN which, besides NAD⁺ and NADP⁺, are important in biology for hydrogen transfer. They are formed from vitamin B2 by phosphorylation (FMN) and subsequent adenylation (FAD).

Vitamin B2 is synthesized in plants, yeasts and many microorganisms from GTP and ribulose 5-phosphate. The reaction pathway starts with opening of the imidazole ring of GTP and elimination of a phosphate residue. Deamination, reduction and elimination of the remaining phosphate result in 5-amino-6-ribitylamino-2,4-pyrimidinone. Reaction of this compound with 3,4-dihydroxy-2-butanone 4-phosphate leads to the bicyclic molecule 6,7-dimethyl-8-ribityllumazine. This compound is converted into the tricyclic compound riboflavin by dismutation, in which a 4-carbon unit is transferred.

Vitamin B2 occurs in many vegetables and in meat, and to a lesser extent in cereal products. The daily vitamin B2 requirement of an adult is about 1.4 to 2 mg. The main breakdown product of the coenzymes FMN and FAD in humans is in turn riboflavin, which is excreted as such.

Vitamin B2 is thus an important dietary substance for humans and animals. Efforts are therefore being made to make vitamin B2 available on the industrial scale. It has therefore been proposed to synthesize vitamin B2 by a microbiological route. Microorganisms which can be used for this purpose are, for example, Bacillus subtilis, the ascomycetes Eremothecium ashbyii, Ashbya gossypii, and the yeasts Candida flari and Saccharomyces cerevisiae. The nutrient media used for this purpose comprise molasses or vegetable oils as carbon source, inorganic salts, amino acids, animal or vegetable peptones and proteins, and vitamin additions. In sterile aerobic submerged processes, yields of more than 10 g of vitamin B2 are obtained per liter of culture broth within a few days. The requirements are good aeration of the culture, careful agitation and setting of temperatures below about 30° C. Removal of the biomass, evaporation and drying of the concentrate result in a product enriched in vitamin B2.

Microbiological production of vitamin B2 is described, for example, in WO-A-92/01060, EP-A-0 405 370 and EP-A-0 531 708.

A survey of the importance, occurrence, production, biosynthesis and use of vitamin B2 is to be found, for example, in Ullmann's Encyclopaedia of Industrial Chemistry, volume A27, pages 521 et seq.

Cell membranes serve a number of functions in a cell. First of all, a membrane demarcates the contents of the cell from the surroundings, so that the cell retains integrity. The membranes also serve as barriers so that dangerous or unwanted compounds cannot flow in and wanted compounds cannot flow out. Cell membranes are, because of their structure, naturally impermeable to the nonfacilitated diffusion of hydrophilic compounds such as proteins, water molecules and ions: a bilayer of lipid molecules in which the polar head groups project toward the outside (out of the cell or into the interior of the cell) and the nonpolar tails project toward the middle of the bilayer and form a hydrophobic core (for a general overview of the structure and function of the membrane, see Gennis, R. B. (1989) Biomembranes, Molecular Structure and Function, Springer: Heidelberg). This barrier makes it possible for cells to contain a relatively larger concentration of wanted compounds and a relatively smaller concentration of unwanted compounds than the surrounding medium, because diffusion of these compounds through the membrane is efficiently blocked.

However, the membrane also provides an effective barrier to the import of wanted molecules and the export of waste molecules. To overcome this difficulty, the cell membranes contain many types of transporter proteins able to facilitate transmembrane transport of various types of compounds: pores or channels and transporters. The former are integral membrane proteins, occasionally protein complexes, which form a regulated aperture through the membrane. This regulation or this “gating” is usually specific for the substrates to be transported through the pores or the channel, so that these transmembrane constructs are specific for a specific class of substrates; for example a potassium channel is constructed in such a way that only ions with a similar charge and size to potassium can pass through. Channel and pore proteins have certain hydrophobic and hydrophilic domains so that the hydrophobic portion of the protein can attach to the inside of the membrane, whereas the hydrophilic portion constitutes the inside of the channel, thus providing a protected hydrophilic environment through which the selected hydrophilic molecule can pass. Many such pores/channels are known in the special field, including those for potassium, calcium, sodium and chloride ions.

This system mediated by pores and channels is restricted to very small molecules, such as ions, because pores or channels sufficiently large for it to be possible for complete proteins to pass through them by facilitated diffusion would not be able to prevent smaller molecules passing through too. Transport of molecules by this process is occasionally referred to as “facilitated diffusion” because the driving force of a concentration gradient is necessary for transport to take place. Permeases likewise enable facilitated diffusion of larger molecules such as glucose or other sugars into the cell when the concentration of these molecules is larger on one side of the membrane than on the other (also referred to as “uniport”). In contrast to pores or channels, these integral proteins (which often have 6 to 14 membrane-spanning helices) do not form open channels through the membrane, but they do bind to the target molecule on the membrane surface and then undergo a conformational change so that the target molecule is released on the opposite side of the membrane.

However, cells often require molecules to be imported or exported against the existing concentration gradient (“active transport”), a situation in which facilitated diffusion cannot take place. There are two general mechanisms used by the cell for such membrane transport: symport or antiport, and energy-coupled transport, such as that mediated by ABC transporters. Symport and antiport systems couple the movement of two different molecules across the membrane (via permeases with two separate binding sites for two different molecules); both molecules are transported in the same direction in symport, whereas one molecule is imported and the other molecule is exported in antiport. This is energetically possible because one of these two molecules moves along a concentration gradient, and this energetically favorable event is made possible only by a simultaneous movement of a required compound against a prevailing concentration gradient. Some molecules can be transported across the membrane against the concentration gradient in an energy-driven process as, for example, with the ABC transporters. In this system, the transport protein located in the membrane has an ATP-binding cassette and, on binding of the target molecule, ATP is converted into ADP+Pi, and the resulting energy which is liberated is used to instigate the movement of the target molecule to the opposite side of the membrane, which is facilitated by the transporter. For more detailed descriptions of all the transport systems, see Bamberg, E. et al., (1993) “Charge transport of ion pumps on lipid bilayer membranes”, Q. Rev.

Biophys. 26: 1-25; Findlay, J. B. C. (1991) “Structure and function in membrane transport systems”, Curr. Opin. Struct. Biol. 1: 804-810; Higgins, C. F. (1992) “ABC transporters from microorganisms to man”, Ann. Rev. Cell. Biol. 8: 67-113; Gennis, R. B. (1989) “Pores, Channels and Transporters”, in Biomembranes, Molecular Structure and Function, Springer: Heidelberg, pages 270-322; and Nikaido, H. and Saier, H. (1992) “Transport proteins in bacteria: common themes in their design”, Science 258: 936-942, and the references present in each of these citations. The utilization of genes of transmembrane transport for generating microorganisms, preferably of the genus Ashbya, in particular of Ashbya gossypii strains, with altered transmembrane transport properties has not yet been described.

It is an object of the present invention to provide novel targets for influencing transmembrane transport in microorganisms of the genus Ashbya, in particular in Ashbya gossypii. The object in particular is to improve transmembrane transport in such microorganisms. A further object is to improve the vitamin B2 production by such microorganisms.

We have found that this object is achieved in particular by providing encoding nucleic acid sequences which are up- or downregulated in Ashbya gossypii during vitamin B2 production (based on results found with the aid of the MPSS analytical method described in detail in the experimental part).

We have found that this object is achieved in particular by providing polynucleotides which can be isolated from Ashbya gossypii and code for a protein which is associated with transmembrane transport and/or is a transmembrane protein, and in particular have a structural (e.g. sequence homology) and/or functional property (e.g. enzymic activity) indicated in table 1; in particular:
a) a, preferably upregulated, nucleic acid sequence which codes for a protein having the function of a mitochondrial energy transfer protein.

In a preferred embodiment of this aspect of the invention there has been isolation of a DNA clone which codes for a characteristic part-sequence of the nucleic acid sequence of the invention and which bears the internal name “Oligo 19”.

In a further preferred embodiment there has been isolation according to the invention of a DNA clone which codes for the complete sequence of the nucleic acid of the invention and which bears the internal name “Oligo 19v”.

One aspect of the present invention relates to a polynucleotide comprising a nucleic acid sequence as shown in SEQ ID NO:1. A further aspect of the invention relates to a polynucleotide comprising a nucleic acid sequence as shown in SEQ ID NO:3 or a fragment thereof. The polynucleotides can be isolated preferably from a microorganism of the genus Ashbya, in particular A. gossypii. The invention additionally relates to the polynucleotides complementary thereto; and to the sequences derived from these polynucleotides through the degeneracy of the genetic code.

The inserts of “Oligo 19” and “Oligo 19v” have significant homologies with the MIPS tag “Ygr257c” from S. cerevisiae. The inserts have a nucleic acid sequence as shown in SEQ ID NO: 1 or SEQ ID NO:3. The amino acid sequence or amino acid part-sequence derived from the corresponding complementary strand of SEQ ID NO:1 or from the encoding strand as shown in SEQ ID NO:3 has significant sequence homology with a mitochondrial energy transfer protein from S. cerevisiae.
b) a, preferably upregulated, nucleic acid sequence which codes for a protein having the function corresponding to that of an ABC transport protein from S. cerevisiae. ABC (ATP binding cassette) proteins function as transport systems and are involved in the uptake or release of substrates from the cells. The transport process is in this case driven by ATP hydrolysis.

In a preferred embodiment of this aspect of the invention there has been isolation of a DNA clone which codes for a characteristic part-sequence of the nucleic acid sequence of the invention and which bears the internal name “Oligo 24”.

In a further preferred embodiment there has been isolation according to the invention of a DNA clone which codes for the complete sequence of the nucleic acid of the invention and which bears the internal name “Oligo 24v”.

One aspect of the present invention relates to a polynucleotide comprising a nucleic acid sequence as shown in SEQ ID NO:5. A further aspect of the invention relates to a polynucleotide comprising a nucleic acid sequence as shown in SEQ ID NO:8 or a fragment thereof. The polynucleotides can be isolated preferably from a microorganism of the genus Ashbya, in particular A. gossypii. The invention additionally relates to the polynucleotides complementary thereto; and to the sequences derived from these polynucleotides through the degeneracy of the genetic code.

The inserts of “Oligo 24” and “Oligo 24v” have significant homologies with the MIPS tag “Mdl2” from S. cerevisiae. The inserts have a nucleic acid sequence as shown in SEQ ID NO:5 or SEQ ID NO:8. The amino acid sequence or amino acid part-sequence derived from the corresponding complementary strand of SEQ ID NO:5 or from the encoding strand as shown in SEQ ID NO:8 has significant sequence homology with an ABC transport protein from S. cerevisiae.
c) a, preferably downregulated, nucleic acid sequence which codes for a protein having the function of a membrane-integrated mitochondrial protein.

In a preferred embodiment of this aspect of the invention there has been isolation of a DNA clone which codes for a characteristic part-sequence of the nucleic acid sequence of the invention and which bears the internal name “Oligo 109”.

In a further preferred embodiment there has been isolation according to the invention of a DNA clone which codes for the complete sequence of the nucleic acid of the invention and which bears the internal name “Oligo 109v”.

A first aspect of the present invention relates to a polynucleotide comprising a nucleic acid sequence as shown in SEQ ID NO: 10. A further aspect of the invention relates to a polynucleotide comprising a nucleic acid sequence as shown in SEQ ID NO:12 or a fragment thereof. The polynucleotides can be isolated preferably from a microorganism of the genus Ashbya, in particular A. gossypii. The invention additionally relates to the polynucleotides complementary thereto; and to the sequences derived from these polynucleotides through the degeneracy of the genetic code.

The inserts of “Oligo 109” and “Oligo 109v” have significant homologies with the MIPS tag “Prp12” from S. cerevisiae. The inserts have a nucleic acid sequence as shown in SEQ ID NO:10 or SEQ ID NO:12. The amino acid sequence or amino acid part-sequence derived from the encoding strand has significant sequence homology with a membrane-integrated mitochondrial protein from S. cerevisiae.
d) a, preferably downregulated, nucleic acid sequence which codes for a protein having the function of a mitochondrial inner membrane transport protein.

In a preferred embodiment of this aspect of the invention there has been isolation of a cDNA clone which codes for a characteristic part-sequence of the nucleic acid sequence of the invention and which bears the internal name “Oligo 163”.

In a further preferred embodiment there has been isolation according to the invention of a DNA clone which codes for the complete sequence of the nucleic acid sequence of the invention and which bears the internal name “Oligo 163v”.

A first aspect of the present invention relates to a polynucleotide comprising a nucleic acid sequence as shown in SEQ ID NO: 14. A further aspect of the invention relates to a polynucleotide comprising a nucleic acid sequence as shown in SEQ ID NO:17 or a fragment thereof. The polynucleotides can be isolated preferably from a microorganism of the genus Ashbya, in particular A. gossypii. The invention additionally relates to the polynucleotides complementary thereto; and to the sequences derived from these polynucleotides through the degeneracy of the genetic code.

The inserts of “Oligo 163” and “Oligo 163v” have significant homologies with the MIPS tag “Flx1” from S. cerevisiae. The inserts have a nucleic acid sequence as shown in SEQ ID NO:14 or SEQ ID NO:17. The amino acid sequence or amino acid part-sequence derived from the encoding strand has significant sequence homology with a mitochondrial inner membrane transport protein from S. cerevisiae.
e) a, preferably downregulated, nucleic acid sequence which codes for a protein having the function of a non-vacuolar 102 kD subunit of the H⁺-ATPase V0 domain.

In a preferred embodiment of this aspect of the invention there has been isolation of a DNA clone which codes for a characteristic part-sequence of the nucleic acid sequence of the invention and which bears the internal name “Oligo 31”.

In a further preferred embodiment there has been isolation according to the invention of a DNA clone which codes for the complete sequence of the nucleic acid of the invention and which bears the internal name “Oligo 31v”.

A first aspect of the present invention relates to a polynucleotide comprising a nucleic acid sequence as shown in SEQ ID NO: 19. A further aspect of the invention relates to a polynucleotide comprising a nucleic acid sequence as shown in SEQ ID NO:21 or a fragment thereof. The polynucleotides can be isolated preferably from a microorganism of the genus Ashbya, in particular A. gossypii. The invention additionally relates to the polynucleotides complementary thereto; and to the sequences derived from these polynucleotides through the degeneracy of the genetic code.

The inserts of “Oligo 31” and “Oligo 31v” have significant homologies with the MIPS tag “STV1” from S. cerevisiae. The inserts have a nucleic acid sequence as shown in SEQ ID NO:19 or SEQ ID NO:21. The amino acid sequence or amino acid part-sequence derived from the encoding strand has significant sequence homology with a non-vacuolar 102 kD subunit of the H⁺-ATPase V0 domain from S. cerevisiae.
f) a, preferably upregulated, nucleic acid sequence which codes for a protein having a function which displays similarity with that of the isp4 protein from S. pombe.

In a preferred embodiment of this aspect of the invention there has been isolation of a cDNA clone which codes for a characteristic part-sequence of the nucleic acid sequence of the invention and which bears the internal name “Oligo 4”.

In a further preferred embodiment there has been isolation according to the invention of a DNA clone which codes for the complete sequence of the nucleic acid of the invention and which bears the internal name “Oligo 4v”.

A first aspect of the present invention relates to a polynucleotide comprising a nucleic acid sequence as shown in SEQ ID NO:23. A further aspect of the invention relates to a polynucleotide comprising a nucleic acid sequence as shown in SEQ ID NO:26 or the sequence complementary thereto as shown in SEQ ID NO:25. The polynucleotides can be isolated preferably from a microorganism of the genus Ashbya, in particular A. gossypii. The invention additionally relates to the polynucleotides complementary thereto; and to the sequences derived from these polynucleotides through the degeneracy of the genetic code.

The inserts of “Oligo 4” and “Oligo 4v” have significant homologies with the MIPS tag “OPT2” from S. cerevisiae. The inserts comprises a nucleic acid sequence as shown in SEQ ID NO:23 or 25. The amino acid sequence or amino acid part-sequence derived from the encoding strand (comprising SEQ ID NO:26) has significant sequence homology with a protein from S. cerevisiae having similarity to the isp4 protein from S. pombe. The proteins of the invention are therefore assigned the activity of an oligopeptide transporter.
g) a, preferably upregulated, nucleic acid sequence which codes for a protein having the function of a VAC1 protein from S. cerevisiae, a cytosolic and peripheral membrane protein having three zinc fingers.

In a preferred embodiment of this aspect of the invention there has been isolation of a DNA clone which codes for a characteristic part-sequence of the nucleic acid sequence of the invention and which bears the internal name “Oligo 6”.

In a further preferred embodiment there has been isolation according to the invention of a DNA clone which codes for the complete sequence of the nucleic acid of the invention and which bears the internal name “Oligo 6v”.

A first aspect of the present invention relates to a polynucleotide comprising a nucleic acid sequence as shown in SEQ ID NO:28. A further aspect of the invention relates to a polynucleotide comprising a nucleic acid sequence as shown in SEQ ID NO:31 or a fragment thereof. The polynucleotides can be isolated preferably from a microorganism of the genus Ashbya, in particular A. gossypii. The invention additionally relates to the polynucleotides complementary thereto; and to the sequences derived from these polynucleotides through the degeneracy of the genetic code.

The inserts of “Oligo 6” and “Oligo 6v” have significant homologies with the MIPS tag “VAC1” from S. cerevisiae. The inserts have a nucleic acid sequence as shown in SEQ ID NO:28 or SEQ ID NO:31. The amino acid sequence or amino acid part-sequence derived from the corresponding complementary strand to SEQ ID NO:28 or from the strand as shown in SEQ ID NO:3 has significant sequence homology with a VAC1 protein, a cytosolic and peripheral membrane protein having three zinc fingers, from S. cerevisiae.
h) a, preferably upregulated, nucleic acid sequence which codes for a protein having an ATPase-like function.

In a preferred embodiment of this aspect of the invention there has been isolation of a DNA clone which codes for a characteristic part-sequence of the nucleic acid sequence of the invention and which bears the internal name “Oligo 146”.

In a further preferred embodiment there has been isolation according to the invention of a DNA clone which codes for the complete sequence of the nucleic acid of the invention and which bears the internal name “Oligo 146v”.

A first aspect of the present invention relates to a polynucleotide comprising a nucleic acid sequence as shown in SEQ ID NO:33. A further aspect of the invention relates to a polynucleotide comprising a nucleic acid sequence as shown in SEQ ID NO:35 or a fragment thereof. The polynucleotides can be isolated preferably from a microorganism of the genus Ashbya, in particular A. gossypii. The invention additionally relates to the polynucleotides complementary thereto; and to the sequences derived from these polynucleotides through the degeneracy of the genetic code.

The inserts of “Oligo 146” and “Oligo 146v” have significant homologies with the MIPS tag “Ymr162c” from S. cerevisiae. The inserts have a nucleic acid sequence as shown in SEQ ID NO:33 or SEQ ID NO:35. The amino acid sequence or amino acid part-sequence derived from the corresponding complementary strand of SEQ ID NO:33 or from the encoding strand as shown in SEQ ID NO:35 has significant sequence homology with a protein having an ATPase or ATPase-like function from S. cerevisiae.
i) a, preferably upregulated, nucleic acid sequence which codes for a protein having the function comparable to that of a PHO85 protein from S. cerevisiae. PHO85 is a kinase and is involved in various cellular processes, including regulation of the PHO gene, glycogen metabolism, regulation of the cell cycle and of cell morphology.

In a preferred embodiment of this aspect of the invention there has been isolation of a DNA clone which codes for a characteristic part-sequence of the nucleic acid sequence of the invention and which bears the internal name “Oligo 56”.

In a further preferred embodiment there has been isolation according to the invention of a DNA clone which codes for the complete sequence of the nucleic acid of the invention and which bears the internal name “Oligo 56v”.

A first aspect of the present invention relates to a polynucleotide comprising a nucleic acid sequence as shown in SEQ ID NO:37. A further aspect of the invention relates to a polynucleotide comprising a nucleic acid sequence as shown in SEQ ID NO:40 or a fragment thereof. The polynucleotides can be isolated preferably from a microorganism of the genus Ashbya, in particular A. gossypii. The invention additionally relates to the polynucleotides complementary thereto; and to the sequences derived from these polynucleotides through the degeneracy of the genetic code.

The inserts of “Oligo 56” and “Oligo 56v” have significant homologies with the MIPS tag “Ypl110c” from S. cerevisiae. The inserts have a nucleic acid sequence as shown in SEQ ID NO:37 or SEQ ID NO:40. The amino acid sequence or amino acid part-sequence derived from the corresponding complementary strand to SEQ ID NO:37 or from the encoding strand as shown in SEQ ID NO:40 has significant sequence homology with a PHO85 protein from S. cerevisiae.
k) a, preferably upregulated, nucleic acid sequence which codes for a protein having the function comparable to that of a S. cerevisiae p24 protein involved in membrane trafficking. Members of the p24 protein family are small type I transmembrane proteins with a short cytoplasmic COOH terminus. They exercise a transport function in the early secretory pathway and are involved, for example, in the transport of various secretory proteins from the endoplasmic reticulum to the Golgi apparatus.

In a preferred embodiment of this aspect of the invention there has been isolation of a DNA clone which codes for a characteristic part-sequence of the nucleic acid sequence of the invention and which bears the internal name “Oligo 167”.

In a further preferred embodiment there has been isolation according to the invention of a DNA clone which codes for the complete sequence of the nucleic acid of the invention and which bears the internal name “Oligo 167v”.

A first aspect of the present invention relates to a polynucleotide comprising a nucleic acid sequence as shown in SEQ ID NO:42. A further aspect of the invention relates to a polynucleotide comprising a nucleic acid sequence as shown in SEQ ID NO:44 or a fragment thereof. The polynucleotides can be isolated preferably from a microorganism of the genus Ashbya, in particular A. gossypii. The invention additionally relates to the polynucleotides complementary thereto; and to the sequences derived from these polynucleotides through the degeneracy of the genetic code.

The inserts of “Oligo 167” and “Oligo 167v” have significant homologies with the MIPS tag “ERP5” from S. cerevisiae. The inserts have a nucleic acid sequence as shown in SEQ ID NO:42 or SEQ ID NO:44. The amino acid sequences derived from the corresponding complementary strand to SEQ ID NO:42 and from the encoding strand of SEQ ID NO:44 have significant sequence homology with the S. cerevisiae p24 protein involved in membrane trafficking.

A further aspect of the invention relates to oligonucleotides which hybridize with one of the above polynucleotides, in particular under stringent conditions.

The invention additionally relates to polynucleotides which hybridize with one of the oligonucleotides of the invention and code for a gene product from microorganisms of the genus Ashbya or a functional equivalent of this gene product.

The invention further relates to polypeptides or proteins which are encoded by the polynucleotides described above; and to peptide fragments thereof which have an amino acid sequence which comprises at least 10 consecutive amino acid residues as shown in SEQ ID NO: 2, 4, 6, 7, 9, 11, 13, 15, 16, 18, 20, 22, 24, 27, 29, 30, 32, 34, 36, 38, 39, 41, 43 or SEQ ID NO:45; and to functional equivalents of the polypeptides or proteins of the invention.

In this connection, functional equivalents differ from the products specifically disclosed in the invention by their amino acid sequence through addition, insertion, substitution, deletion or inversion at a minimum of one, such as, for example, 1 to 30 or 1 to 20 or 1 to 10, sequence positions without the originally observed protein function, which can be deduced by sequence comparison with other proteins, being lost. It is thus possible for equivalents to have essentially identical, higher or lower activities compared with the native protein.

Further aspects of the invention relate to expression cassettes for the recombinant production of proteins of the invention, comprising one of the nucleic acid sequences defined above, operatively linked to at least one regulatory nucleic acid sequence; and to recombinant vectors comprising at least one such expression cassette of the invention.

Also provided according to the invention are prokaryotic or eukaryotic hosts which are transformed with at least one vector of the above type. A preferred embodiment provides prokaryotic or eukaryotic hosts in which the functional expression of at least one gene which codes for a polypeptide of the invention as defined above is modulated (e.g. inhibited or overexpressed); or in which the biological activity of a polypeptide as defined above is reduced or increased. Preferred hosts are selected from ascomycetes, in particular those of the genus Ashbya and preferably strains of A. gossypii.

Modulation of gene expression in the above sense includes both inhibition thereof, for example through blockade of a stage in expression (in particular transcription or translation) or a specific overexpression of a gene (for example through modification of regulatory sequences or increasing the copy number of the coding sequence).

A further aspect of the invention relates to the use of an expression cassette of the invention, of a vector of the invention or of a host of the invention for the microbiological production of vitamin B2 and/or precursors and/or derivatives thereof.

A further aspect of the invention relates to the use of an expression cassette of the invention, of a vector of the invention or of a host of the invention for the recombinant production of a polypeptide of the invention as defined above.

Also provided according to the invention is a method for detecting or for validating an effector target for modulating the microbiological production of vitamin B2 and/or precursors and/or derivatives thereof. This entails treating a microorganism capable of the microbiological production of vitamin B2 and/or precursors and/or derivatives thereof with an effector which interacts with (such as, for example, non-covalently binds to) a target selected from a polypeptide of the invention as defined above or a nucleic acid sequence coding therefor, validating the influence of the effector on the amount of the microbiologically produced vitamin B2 and/or of the precursor and/or of a derivative thereof; and isolating the target where appropriate. The validation in this case takes place preferably by direct comparison with the microbiological vitamin B2 production in the absence of the effector under otherwise identical conditions.

A further aspect of the invention relates to a method for modulating (in relation to the amount and/or rate of) the microbiological production of vitamin B2 and/or precursors and/or derivatives thereof, where a microorganism capable of the microbiological production of vitamin B2 and/or precursors and/or derivatives thereof is treated with an effector which interacts with a target selected from a polypeptide of the invention as defined above or a nucleic acid sequence coding therefor.

Preferred examples of the abovementioned effectors which should be mentioned are:
a) antibodies or antigen-binding fragments thereof;
b) polypeptide ligands which are different from a) and which interact with a polypeptide of the invention;
c) low molecular weight effectors which modulate the biological activity of a polypeptide of the invention;
d) antisense nucleic acid sequences which interact with a nucleic acid sequence of the invention.

The invention likewise relates to abovementioned effectors having specificity for at least one of the targets, according to the invention, defined above.

A further aspect of the invention relates to a method for the microbiological production of vitamin B2 and/or precursors and/or derivatives thereof, where a host as defined above is cultivated under conditions favoring the production of vitamin B2 and/or precursors and/or derivatives thereof, and the desired product(s) is(are) isolated from the culture mixture. It is preferred in this connection that the host is treated with an effector as defined above before and/or during the cultivation. A preferred host is in this case selected from microorganisms of the genus Ashbya; in particular transformed as described above.

A final aspect of the invention relates to the use of a polynucleotide or polypeptide of the invention as target for modulating the production of vitamin B2 and/or precursors and/or derivatives thereof in a microorganism of the genus Ashbya.

### DESCRIPTION OF THE FIGURES

FIG. 1 shows an alignment between an amino acid part-sequence of the invention (corresponding to the complementary strand to position 609 to 1 in SEQ ID NO:1) (upper sequence) and a part-sequence of the MIPS tag “Ygr257c” from S. cerevisiae (lower sequence). Identical sequence positions are indicated between the two sequences. Similar sequence positions are labeled with “+”.

FIG. 2 shows an alignment between an amino acid part-sequence of the invention (SEQ ID NO:6) (corresponding to the complementary strand to position 1494 to 1387 SEQ ID NO:5) (upper sequence) and a part-sequence of the MIPS tag Mdl2 from S. cerevisiae (lower sequence). Identical sequence positions are indicated between the two sequences. Similar sequence positions are labeled with “+”.

FIG. 3 shows an alignment between an amino acid part-sequence of the invention (corresponding to the coding strand in position 15 to 455 in SEQ ID NO:10) (upper sequence) and a part-sequence of the MIPS tag Prp12 from S. cerevisiae (lower sequence). Identical sequence positions are indicated between the two sequences. Similar sequence positions are labeled with “+”.

FIG. 4 shows an alignment between an amino acid part-sequence of the invention (corresponding to the coding strand in position 246 to 1118 in SEQ ID NO:14) (upper sequence) and a part-sequence of the MIPS tag Flx1 from S. cerevisiae (lower sequence). Identical sequence positions are indicated between the two sequences. Similar sequence positions are labeled with “+”.

FIG. 5 shows an alignment between an amino acid part-sequence of the invention (corresponding to the coding strand in position 2 to 790 in SEQ ID NO:19) (upper sequence) and a part-sequence of the MIPS tag STV1 from S. cerevisiae (lower sequence). Identical sequence positions are indicated between the two sequences. Similar sequence positions are labeled with “+”.

FIG. 6 shows an alignment between an amino acid part-sequence of the invention (corresponding to the complementary strand to position 869 to 522 in SEQ ID NO:23) (upper sequence) and a part-sequence of the MIPS tag OPT2 from S. cerevisiae (lower sequence). Identical sequence positions are indicated between the two sequences. Similar sequence positions are labeled with “+”.

FIG. 7A shows an alignment between an amino acid part-sequence of the invention (corresponding to the complementary strand to position 356 to 243 in SEQ ID NO:28) (upper sequence) and a part-sequence of the MIPS tag VAC1 from S. cerevisiae (lower sequence). Identical sequence positions are indicated between the two sequences. Similar sequence positions are labeled with “+”. FIG. 7B shows an alignment between an amino acid part-sequence of the invention (corresponding to the complementary strand to position 166 to 2 in SEQ ID NO:28) (upper sequence) and a part-sequence of the MIPS tag VAC1 from S. cerevisiae (lower sequence). Identical sequence positions are indicated between the two sequences. Similar sequence positions are labeled with “+”.

FIG. 8 shows an alignment between an amino acid part-sequence of the invention (corresponding to the complementary strand to position 904 to 707 in SEQ ID NO:33) (upper sequence) and a part-sequence of the MIPS tag Ymr162c from S. cerevisiae (lower sequence). Identical sequence positions are indicated between the two sequences. Similar sequence positions are labeled with “+”.

FIG. 9 shows an alignment between an amino acid part-sequence of the invention (corresponding to the complementary strand to position 898 to 5 in SEQ ID NO:37) (upper sequence) and a part-sequence of the MIPS tag Ypl110c from S. cerevisiae (lower sequence). Identical sequence positions are indicated between the two sequences. Similar sequence positions are labeled with “+”.

FIG. 10 shows an alignment between an amino acid part-sequence of the invention (corresponding to the complementary strand to position 931 to 806 in SEQ ID NO:42) (upper sequence) and a part-sequence of the MIPS tag ERP5 from S. cerevisiae (lower sequence). Identical sequence positions are indicated between the two sequences. Similar sequence positions are labeled with “+”.

### DETAILED DESCRIPTION OF THE INVENTION

The nucleic acid molecules of the invention encode polypeptides or proteins which are referred to here as proteins of transmembrane transport (for example with activity in relation to transmembrane transport systems) or for short as “TMT proteins”. These TMT proteins have, for example, a function in the control of membrane-associated transporter systems which transport required proteins with consumption of energy against a concentration gradient into the cell. The TMT proteins are able to influence the cellular response to external conditions and thus for example regulate the metabolism of the cell. Owing to the availability of cloning vectors which can be used in Ashbya gossypii, as disclosed, for example, in Wright and Philipsen (1991) Gene, 109, 99-105, and of techniques for genetic manipulation of A. gossypii and the related yeast species, the nucleic acid molecules of the invention can be used for genetic manipulation of these organisms, in particular of A. gossypii, in order to make them better and more efficient producers of vitamin B2 and/or precursors and/or derivatives thereof. This improved production or efficiency may result from a direct effect of the manipulation of a gene of the invention or result from an indirect effect of such a manipulation.

The present invention is based on the provision of novel molecules which are referred to here as TMT nucleic acids and TMT proteins and are involved in transmembrane transport, in particular in Ashbya gossypii (e.g. in the synthesis or regulation of transport proteins). The activity of the TMT molecules of the invention in A. gossypii influences vitamin B2 production by this organism. The activity of the TMT molecules of the invention is preferably modulated so that the metabolic and/or energy pathways of A. gossypii in which the TMT proteins of the invention are involved are modulated in relation to the yield, production and/or efficiency of vitamin B2 production, which modulates either directly or indirectly the yield, production and/or efficiency of vitamin B2 production in A. gossypii.

The nucleic acid sequences provided by the invention can be isolated, for example, from the genome of an Ashbya gossypii strain which is freely available from the American Type Culture Collection under the number ATCC 10895.

Improvement in vitamin B2 Production:

There is a number of possible mechanisms by which the yield, production and/or efficiency of production of vitamin B2 by an A. gossypii strain can be influenced directly through changing the amount and/or activity of a TMT protein of the invention.

Thus, a more efficient transmembrane transport enables the cellular response to be enhanced, and thus the formation of the desired products of value to be increased. Mutagenesis of one or more TMT proteins of the invention may also lead to TMT proteins with altered (increased or reduced) activities which influence indirectly the production of the required product from A. gossypii. It is possible, for example, with the aid of the TMT proteins to adapt the cells to new or altered external conditions. It is possible, by improving the growth and multiplication of these modified cells, to increase the viability of the cells in larger-scale cultures and also to improve the rate of division.

Finally, it is possible thereby to increase the yield of desired target products produced by these cells.

Polypeptides:

The invention relates to polypeptides which comprise the abovementioned amino acid sequences or characteristic part-sequences thereof and/or are encoded by the nucleic acid sequences described herein.

The invention likewise encompasses “functional equivalents” of the specifically disclosed novel polypeptides.

“Functional equivalents” or analogs of the specifically disclosed polypeptides are for the purposes of the present invention polypeptides which differ therefrom but which still have the desired biological activity (such as, for example, substrate specificity).

“Functional equivalents” mean according to the invention in particular mutants which have in at least one of the abovementioned sequence positions an amino acid which differs from that specifically mentioned but nevertheless have one of the abovementioned biological activities. “Functional equivalents” thus comprise the mutants obtainable by one or more amino acid additions, substitutions, deletions and/or inversions, it being possible for said modifications to occur in any sequence position as long as they lead to a mutant having the profile of properties of the invention. Functional equivalence exists in particular also when there is qualitative agreement between mutant and unmodified polypeptide in the reactivity patterns, i.e. there are differences in the rate of conversion of identical substrates, for example.

“Functional equivalents” in the above sense are also precursors of the polypeptides described, and functional derivatives and salts of the polypeptides. The term “salts” means both salts of carboxyl groups and acid addition salts of amino groups in the protein molecules of the invention. Salts of carboxyl groups can be prepared in a manner known per se and comprise inorganic salts such as, for example, sodium, calcium, ammonium, iron and zinc salts, and salts with organic bases such as, for example, amines such as triethanolamine, arginine, lysine, piperidine and the like. Acid addition salts such as, for example, salts with mineral acids such as hydrochloric acid or sulfuric acid and salts with organic acids such as acetic acid and oxalic acid are also an aspect of the invention.

“Functional derivatives” of polypeptides of the invention can also be prepared at functional amino acid side groups or at their N- or C-terminal end by known techniques. Such derivatives include for example aliphatic esters of carboxyl groups, amides of carboxyl groups obtainable by reaction with ammonia or with a primary or secondary amine; N-acyl derivatives of free amino groups prepared by reaction with acyl groups; or O-acyl derivatives of free hydroxyl groups prepared by reaction with acyl groups.

“Functional equivalents” naturally also comprise polypeptides which are obtainable from other organisms, and naturally occurring variants. For example, homologous sequence regions can be found by sequence comparison, and equivalent enzymes can be established on the basis of the specific requirements of the invention.

“Functional equivalents” likewise comprise fragments, preferably single domains or sequence motifs, of the polypeptides of the invention, which have, for example, the desired biological function.

“Functional equivalents” are additionally fusion proteins which have one of the abovementioned polypeptide sequences or functional equivalents derived therefrom and at least one other heterologous sequence functionally different therefrom in functional N- or C-terminal linkage (i.e. with negligible mutual impairment of the functions of the parts of the fusion proteins). Nonlimiting examples of such heterologous sequences are, for example, signal peptides, enzymes, immunoglobulins, surface antigens, receptors or receptor ligands.

“Functional equivalents” include according to the invention homologs of the specifically disclosed proteins. These have at least 60%, preferably at least 75%, in particular at least 85%, such as, for example, 90%, 95% or 99%, homology to one of the specifically disclosed sequences, calculated by the algorithm of Pearson and Lipman, Proc. Natl. Acad. Sci. (USA) 85(8),1988, 2444-2448.

In the case where protein glycosylation is possible, equivalents of the invention include proteins of the type defined above in deglycosylated or glycosylated form, and modified forms obtainable by altering the glycosylation pattern.

Homologs of the proteins or polypeptides of the invention can be generated by mutagenesis, for example by point mutation or truncation of the protein. The term “homolog” as used here relates to a variant form of the protein which acts as agonist or antagonist of the protein activity.

Homologs of the proteins of the invention can be identified by screening combinatorial libraries of mutants such as, for example, truncation mutants. It is possible, for example, to generate a variegated library of protein variants by combinatorial mutagenesis at the nucleic acid level, such as, for example, by enzymatic ligation of a mixture of synthetic oligonucleotides. There is a large number of methods which can be used to produce libraries of potential homologs from a degenerate oligonucleotide sequence. Chemical synthesis of a degenerate gene sequence can be carried out in an automatic DNA synthesizer, and the synthetic gene can then be ligated into a suitable expression vector. The use of a degenerate set of genes makes it possible to provide all sequences which encode the desired set of potential protein sequences in one mixture. Methods for synthesizing degenerate oligonucleotides are known to the skilled worker (for example Narang, S. A. (1983) Tetrahedron 39: 3; Itakura et al. (1984) Annu. Rev. Biochem. 53: 323; Itakura et al., (1984) Science 198: 1056; Ike et al. (1983) Nucleic Acids Res. 11: 477).

In addition, libraries of fragments of the protein codon can be used to generate a variegated population of protein fragments for screening and for subsequent selection of homologs of a protein of the invention. In one embodiment, a library of coding sequence fragments can be generated by treating a double-stranded PCR fragment of a coding sequence with a nuclease under conditions under which nicking takes place only about once per molecule, denaturing the double-stranded DNA, renaturing the DNA to form double-stranded DNA, which may comprise sense/antisense pairs of different nicked products, removing single-stranded sections from newly formed duplices by treatment with S1 nuclease and ligating the resulting fragment library into an expression vector. It is possible by this method to derive an expression library which encodes N-terminal, C-terminal and internal fragments having different sizes of the protein of the invention.

Several techniques are known in the prior art for screening gene products from combinatorial libraries which have been produced by point mutations or truncation and for screening cDNA libraries for gene products with a selected property. These techniques can be adapted to rapid screening of gene libraries which have been generated by combinatorial mutagenesis of homologs of the invention. The most frequently used techniques for screening large gene libraries undergoing high-throughput analysis comprise the cloning of the gene library into replicable expression vectors, transformation of suitable cells with the resulting vector library and expression of the combinatorial genes under conditions under which detection of the required activity facilitates isolation of the vector which encodes the gene whose product has been detected. Recursive ensemble mutagenesis (REM), a technique which increases the frequency of functional mutants in the libraries, can be used in combination with the screening tests for identifying homologs (Arkin and Yourvan (1992) PNAS 89: 7811-7815; Delgrave et al. (1993) Protein Engineering 6(3): 327-331).

Recombinant preparation of polypeptides of the invention is possible (see following sections) or they can be isolated in native form from microorganisms, especially those of the genus Ashbya, by use of conventional biochemical techniques (see Cooper, T. G., Biochemische Arbeitsmethoden, Verlag Walter de Gruyter, Berlin, New York or in Scopes, R., Protein Purification, Springer Verlag, New York, Heidelberg, Berlin).

Nucleic Acid Sequences:

The invention also relates to nucleic acid sequences (single- and double-stranded DNA and RNA sequences such as, for example, cDNA and mRNA), coding for one of the above polypeptides and their functional equivalents which are obtainable, for example, by use of artificial nucleotide analogs.

The invention relates both to isolated nucleic acid molecules which code for polypeptides or proteins of the invention or biologically active sections thereof, and to nucleic acid fragments which can be used, for example, for use as hybridization probes or primers for identifying or amplifying coding nucleic acids of the invention.

The nucleic acid molecules of the invention may additionally comprise untranslated sequences from the 3′ and/or 5′ end of the coding region of the gene.

An “isolated” nucleic acid molecule is separated from other nucleic acid molecules which are present in the natural source of the nucleic acid and may moreover be essentially free of other cellular material or culture medium if it is produced by recombinant techniques, or free of chemical precursors or other chemicals if it is chemically synthesized.

A nucleic acid molecule of the invention can be isolated by using standard techniques of molecular biology and the sequence information provided according to the invention. For example, cDNA can be isolated from a suitable cDNA library by using one of the specifically disclosed complete sequences or a section thereof as hybridization probe and standard hybridization techniques (as described, for example, in Sambrook, J., Fritsch, E. F. and Maniatis, T. Molecular Cloning: A Laboratory Manual. 2nd edition, Cold Spring Harbor Laboratory, Cold Spring Harbor Laboratory Press, Cold Spring Harbor, N.Y., 1989). It is moreover possible for a nucleic acid molecule comprising one of the disclosed sequences or a section thereof to be isolated by polymerase chain reaction using the oligonucleotide primers constructed on the basis of this sequence.

The nucleic acid amplified in this way can be cloned into a suitable vector and be characterized by DNA sequence analysis. The oligonucleotides of the invention which correspond to a TMT nucleotide sequence can also be produced by standard synthetic methods, for example using an automatic DNA synthesizer.

The invention additionally comprises the nucleic acid molecules which are complementary to the specifically described nucleotide sequences, or a section thereof.

The nucleotide sequences of the invention make it possible to generate probes and primers which can be used for identifying and/or cloning homologous sequences in other cell types and organisms. Such probes and primers usually comprise a nucleotide sequence region which hybridizes under stringent conditions onto at least about 12, preferably at least about 25, such as, for example, about 40, 50 or 75, consecutive nucleotides of a sense strand of a nucleic acid sequence of the invention or a corresponding antisense strand.

Further nucleic acid sequences of the invention are derived from SEQ ID NO: 1, 3, 5, 8, 10, 12, 14, 17, 19, 21, 23, 25, 26, 28, 31, 33, 35, 37, 40, 42 or SEQ ID NO:44 and differ therefrom through addition, substitution, insertion or deletion of one or more nucleotides, but still code for polypeptides having the desired profile of properties.

The invention also encompasses nucleic acid sequences which comprise so-called silent mutations or are modified, by comparison with a specifically mentioned sequence, in accordance with the codon usage of a specific source or host organism, as well as naturally occurring variants such as, for example, splice variants or allelic variants, thereof. It likewise relates to sequences which are obtainable by conservative nucleotide substitutions (i.e. the relevant amino acid is replaced by an amino acid with the same charge, size, polarity and/or solubility).

The invention also relates to molecules derived from the specifically disclosed nucleic acids through sequence polymorphisms. These genetic polymorphisms may exist because of the natural variation between individuals within a population. These natural variations normally result in a variance of from 1 to 5% in the nucleotide sequence of a gene.

The invention additionally encompasses nucleic acid sequences which hybridize with or are complementary to the abovementioned coding sequences. These polynucleotides can be found on screening of genomic or cDNA libraries and, where appropriate, be amplified therefrom by means of PCR using suitable primers, and then, for example, be isolated with suitable probes. Another possibility is to transform suitable microorganisms with polynucleotides or vectors of the invention, multiply the microorganisms and thus the polynucleotides, and then isolate them. An additional possibility is to synthesize polynucleotides of the invention by chemical routes.

The property of being able to “hybridize” onto polynucleotides means the ability of a polynucleotide or oligonucleotide to bind under stringent conditions to an almost complementary sequence, while there are no nonspecific bindings between noncomplementary partners under these conditions. For this purpose, the sequences should be 70-100%, preferably 90-100%, complementary. The property of complementary sequences being able to bind specifically to one another is made use of, for example, in the Northern or Southern blot technique or in PCR or RT-PCR in the case of primer binding. Oligonucleotides with a length of 30 base pairs or more are normally employed for this purpose. Stringent conditions mean, for example, in the Northern blot technique the use of a washing solution at 50-70° C., preferably 60-65° C., for example 0.1×SSC buffer with 0.1% SDS (20×SSC: 3M NaCl, 0.3M Na citrate, pH 7.0) for eluting nonspecifically hybridized cDNA probes or oligonucleotides. In this case, as mentioned above, only nucleic acids with a high degree of complementarity remain bound to one another. The setting up of stringent conditions is known to the skilled worker and is described, for example, in Ausubel et al., Current Protocols in Molecular Biology, John Wiley & Sons, N.Y. (1989), 6.3.1-6.3.6.

A further aspect of the invention relates to antisense nucleic acids. This comprises a nucleotide sequence which is complementary to a coding sense nucleic acid. The antisense nucleic acid may be complementary to the entire coding strand or only to a section thereof. In a further embodiment, the antisense nucleic acid molecule is antisense to a noncoding region of the coding strand of a nucleotide sequence. The term “noncoding region” relates to the sequence sections which are referred to as 5′- and 3′-untranslated regions. An antisense oligonucleotide may be, for example, about 5, 10, 15, 20, 25, 30, 35, 40, 45 or 50 nucleotides long. An antisense nucleic acid of the invention can be constructed by chemical synthesis and enzymatic ligation reactions using methods known in the art.

An antisense nucleic acid can be synthesized chemically, using naturally occurring nucleotides or variously modified nucleotides which are configured so that they increase the biological stability of the molecules or increase the physical stability of the duplex formed between the antisense and sense nucleic acids. Examples which can be used are phosphorothioate derivatives and acridine-substituted nucleotides. Examples of modified nucleosides which can be used for generating the antisense nucleic acid are, inter alia, 5-fluorouracil, 5-bromouracil, 5-chlorouracil, 5-iodouracil, hypoxanthine, xanthine, 4-acetylcytosine, 5-(carboxyhydroxymethyl)uracil, 5-carboxy-methylaminomethyl-2-thiouridine, 5-carboxymethylaminomethyluracil, dihydrouracil, beta-D-galactosylqueuosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methyl-aminomethyluracil, 5-methoxyaminomethyl-2-thiouracil, beta-D-mannosylqueuosine, 5-methoxycarboxymethyluracil, 5-methoxyuracil, 2-methylthio-N-6-isopentenyladenine, uracil-5-oxyacetic acid (v), wybutoxosine, pseudouracil, queuosine, 2-thiocytosine, 5-methyl-2-thiouracil, 2-thiouracil, 4-thiouracil, 5-methyluracil, methyl uracil-5-oxyacetate, 3-(3-amino-3-carboxypropyl)uracil, (acp3)w and 2,6-diaminopurine. The antisense nucleic acid may also be produced biologically by using an expression vector into which a nucleic acid has been subcloned in the antisense direction.

The antisense nucleic acid molecules of the invention are normally administered to a cell or generated in situ so that they hybridize with the cellular mRNA and/or a coding DNA or bind thereto, so that expression of the protein is inhibited for example by inhibition of transcription and/or translation.

The antisense molecule can be modified so that it binds specifically to a receptor or to an antigen which is expressed on a selected cell surface, for example through linkage of the antisense nucleic acid molecule to a peptide or an antibody which binds to a cell surface receptor or antigen. The antisense nucleic acid molecule can also be administered to cells by using the vectors described herein. The vector constructs preferred for achieving adequate intracellular concentrations of the antisense molecules are those in which the antisense nucleic acid molecule is under the control of a strong bacterial, viral or eukaryotic promoter.

In a further embodiment, the antisense nucleic acid molecule of the invention is an alpha-anomeric nucleic acid molecule. An alpha-anomeric nucleic acid molecule forms specific double-stranded hybrids with complementary RNA, with the strands running parallel to one another, in contrast to normal alpha units (Gaultier et al., (1987) Nucleic Acids Res. 15: 6625-6641). The antisense nucleic acid molecule may additionally comprise a 2′-O-methylribonucleotide (Inoue et al., (1987) Nucleic Acids Res. 15: 6131-6148) or a chimeric RNA-DNA analog (Inoue et al. (1987) FEBS Lett. 215: 327-330).

The invention also relates to ribozymes. These are catalytic RNA molecules with ribonuclease activity which are able to cleave a single-stranded nucleic acid such as an mRNA to which they have a complementary region. It is thus possible to use ribozymes (for example hammerhead ribozymes (described in Haselhoff and Gerlach (1988) Nature 334: 585-591)) for the catalytic cleavage of transcripts of the invention in order thereby to inhibit the translation of the corresponding nucleic acid. A ribozyme with specificity for a coding nucleic acid of the invention can be formed, for example, on the basis of a cDNA specifically disclosed herein. For example, a derivative of a tetrahymena-L-19 IVS RNA can be constructed, with the nucleotide sequence of the active site being complementary to the nucleotide sequence to be cleaved in a coding mRNA of the invention. (Compare, for example, U.S. Pat. No. 4,987,071 and U.S. Pat. No. 5,116,742).

Alternatively, mRNA can be used for selecting a catalytic RNA with specific ribonuclease activity from a pool of RNA molecules (see, for example, Bartel, D., and Szostak, J. W. (1993) Science 261: 1411-1418).

Gene expression of sequences of the invention can alternatively be inhibited by targeting nucleotide sequences which are complementary to the regulatory region of a nucleotide sequence of the invention (for example to a promoter and/or enhancer of a coding sequence) so that there is formation of triple helix structures which prevent transcription of the corresponding gene in target cells (Helene, C. (1991) Anticancer Drug Res. 6(6) 569-584; Helene, C. et al., (1992) Ann. N.Y. Acad. Sci. 660: 27-36; and Maher., L. J. (1992) Bioassays 14(12): 807-815).

Expression Constructs and Vectors:

The invention additionally relates to expression constructs comprising, under the genetic control of regulatory nucleic acid sequences, a nucleic acid sequence coding for a polypeptide of the invention; and to vectors comprising at least one of these expression constructs. Such constructs of the invention preferably comprise a promoter 5′-upstream from the particular coding sequence, and a terminator sequence 3′-downstream, and, where appropriate, other usual regulatory elements, in particular each operatively linked to the coding sequence. “Operative linkage” means the sequential arrangement of promoter, coding sequence, terminator and, where appropriate, other regulatory elements in such a way that each of the regulatory elements is able to comply with its function as intended for expression of the coding sequence. Examples of sequences which can be operatively linked are targeting sequences and enhancers, polyadenylation signals and the like. Other regulatory elements comprise selectable markers, amplification signals, origins of replication and the like. Suitable regulatory sequences are described, for example, in Goeddel, Gene Expression Technology: Methods in Enzymology 185, Academic Press, San Diego, Calif. (1990).

In addition to the artificial regulatory sequences it is possible for the natural regulatory sequence still to be present in front of the actual structural gene. This natural regulation can, where appropriate, be switched off by genetic modification, and expression of the genes can be increased or decreased. The gene construct can, however, also have a simpler structure, that is to say no additional regulatory signals are inserted in front of the structural gene, and the natural promoter with its regulation is not deleted. Instead, the natural regulatory sequence is mutated so that regulation no longer takes place, and gene expression is enhanced or diminished. The nucleic acid sequences may be present in one or more copies in the gene construct.

Examples of promoters which can be used are: cos, tac, trp, tet, trp-tet, lpp, lac, lpp-lac, laclq, T7, T5, T3, gal, trc, ara, SP6, λ-PR or λ-PL promoter, which are advantageously used in Gram-negative bacteria; and the Gram-positive promoters amy and SPO2, the yeast promoters ADC1, MFα, AC, P-60, CYC1, GAPDH or the plant promoters CaMV/35S, SSU, OCS, lib4, usp, STLS1, B33, not or the ubiquitin or phaseolin promoter. The use of inducible promoters is particularly preferred, such as, for example, light- and, in particular, temperature-inducible promoters such as the PᵣPₗ promoter. It is possible in principle for all natural promoters with their regulatory sequences to be used. In addition, it is also possible advantageously to use synthetic promoters.

Said regulatory sequences are intended to make specific expression of the nucleic acid sequences possible. This may mean, for example, depending on the host organism, that the gene is expressed or overexpressed only after induction or that it is immediately expressed and/or overexpressed.

The regulatory sequences or factors may moreover preferably influence positively, and thus increase or reduce, expression. Thus, enhancement of the regulatory elements can take place advantageously at the level of transcription by using strong transcription signals such as promoters and/or enhancers. However, it is also possible to enhance translation by, for example, improving the stability of the mRNA.

An expression cassette is produced by fusing a suitable promoter to a suitable enocoding nucleotide sequence and to a terminator signal or polyadenylation signal. Conventional techniques of recombination and cloning are used for this purpose, as described, for example, in T. Maniatis, E. F. Fritsch and J. Sambrook, Molecular Cloning: A Laboratory Manual, Cold Spring Harbor Laboratory, Cold Spring Harbor, N.Y. (1989) and in T. J. Silhavy, M. L. Berman and L. W. Enquist, Experiments with Gene Fusions, Cold Spring Harbor Laboratory, Cold Spring Harbor, N.Y. (1984) and in Ausubel, F. M. et al., Current Protocols in Molecular Biology, Greene Publishing Assoc. and Wiley Interscience (1987).

For expression in a suitable host organism, the recombinant nucleic acid construct or gene construct is advantageously inserted into a host-specific vector, which makes optimal expression of the genes in the host possible. Vectors are well known to the skilled worker and can be found, for example, in “Cloning Vectors” (Pouwels P. H. et al., eds, Elsevier, Amsterdam-New York-Oxford, 1985). Vectors also mean not only plasmids but also all other vectors known to the skilled worker, such as, for example, phages, viruses, such as SV40, CMV, baculovirus and adenovirus, transposons, IS elements, phasmids, cosmids, and linear or circular DNA. These vectors may undergo autonomous replication in the host organism or chromosomal replication.

Examples of suitable expression vectors which may be mentioned are:

Conventional fusion expression vectors such as pGEX (Pharmacia Biotech Inc; Smith, D. B. and Johnson, K. S. (1988) Gene 67: 3140), PMAL (New England Biolabs, Beverly, Mass.) and pRIT 5 (Pharmacia, Piscataway, N.J.), with which respectively glutathione S-transferase (GST), maltose E-binding protein and protein A are fused to the recombinant target protein.

Nonfusion protein expression vectors such as pTrc (Amann et al., (1988) Gene 69: 301-315) and pET 11d (Studier et al. Gene Expression Technology: Methods in Enzymology 185, Academic Press, San Diego, Calif. (1990) 60-89).

Yeast expression vector for expression in the yeast S. cerevisiae, such as pYepSec1 (Baldari et al., (1987) Embo J. 6: 229-234), pMF (Kurjan and Herskowitz (1982) Cell 30: 933-943), pJRY88 (Schultz et al. (1987) Gene 54: 113-123) and pYES2 (Invitrogen Corporation, San Diego, Calif.). Vectors and methods for constructing vectors suitable for use in other fungi such as filamentous fungi comprise those which are described in detail in: van den Hondel, C. A. M. J. J. & Punt, P. J. (1991) “Gene transfer systems and vector development for filamentous fungi, in: Applied Molecular Genetics of Fungi, J. F. Peberdy et al., eds, pp. 1-28, Cambridge University Press: Cambridge.

Baculovirus vectors which are available for expression of proteins in cultured insect cells (for example Sf9 cells) comprise the pAc series (Smith et al., (1983) Mol. Cell Biol. 3: 2156-2165) and pVL series (Lucklow and Summers (1989) Virology 170: 31-39).

Plant expression vectors such as those described in detail in: Becker, D., Kemper, E., Schell, J. and Masterson, R. (1992) “New plant binary vectors with selectable markers located proximal to the left border”, Plant Mol. Biol. 20: 1195-1197; and Bevan, M. W. (1984) “Binary Agrobacterium vectors for plant transformation”, Nucl. Acids Res. 12: 8711-8721.

Mammalian expression vectors such as pCDM8 (Seed, B. (1987) Nature 329: 840) and pMT2PC (Kaufman et al. (1987) EMBO J. 6: 187-195).

Further suitable expression systems for prokaryotic and eukaryotic cells are described in chapters 16 and 17 of Sambrook, J., Fritsch, E. F. and Maniatis, T., Molecular cloning: A Laboratory Manual, 2nd edition, Cold Spring Harbor Laboratory, Cold Spring Harbor Laboratory Press, Cold Spring Harbor, N.Y., 1989.

Recombinant Microorganisms:

The vectors of the invention can be used to produce recombinant microorganisms which are transformed, for example, with at least one vector of the invention and can be employed for producing the polypeptides of the invention. The recombinant constructs of the invention described above are advantageously introduced and expressed in a suitable host system. Cloning and transfection methods familiar to the skilled worker, such as, for example, coprecipitation, protoplast fusion, electroporation, retroviral transfection and the like, are preferably used to bring about expression of said nucleic acids in the particular expression system. Suitable systems are described, for example, in Current Protocols in Molecular Biology, F. Ausubel et al., eds, Wiley Interscience, New York 1997, or Sambrook et al. Molecular Cloning: A Laboratory Manual, 2nd edition, Cold Spring Harbor Laboratory, Cold Spring Harbor Laboratory Press, Cold Spring Harbor, N.Y., 1989.

It is also possible according to the invention to produce homologously recombined microorganisms. This entails production of a vector which contains at least one section of a gene of the invention or a coding sequence, in which, where appropriate, at least one amino acid deletion, addition or substitution has been introduced in order to modify, for example functionally disrupt, the sequence of the invention (knockout vector). The introduced sequence may, for example, also be a homolog from a related microorganism or be derived from a mammalian, yeast or insect source. The vector used for homologous recombination may alternatively be designed so that the endogenous gene is mutated or otherwise modified during the homologous recombination but still encodes the functional protein (for example the regulatory region located upstream may be modified in such a way that this modifies expression of the endogenous protein). The modified section of the TMT gene is in the homologous recombination vector. The construction of suitable vectors for homologous recombination is, for example, described in Thomas, K. R. and Capecchi, M. R. (1987) Cell 51: 503.

Suitable host organisms are in principle all organisms which enable expression of the nucleic acids of the invention, their allelic variants, their functional equivalents or derivatives. Host organisms mean, for example, bacteria, fungi, yeasts, plant or animal cells. Preferred organisms are bacteria, such as those of the genera Escherichia, such as, for example, Escherichia coli, Streptomyces, Bacillus or Pseudomonas, eukaryotic microorganisms such as Saccharomyces cerevisiae, Aspergillus, higher eukaryotic cells from animals or plants, for example Sf9 or CHO cells. Preferred organisms are selected from the genus Ashbya, in particular from A. gossypii strains.

Successfully transformed organisms can be selected through marker genes which are likewise present in the vector or in the expression cassette. Examples of such marker genes are genes for antibiotic resistance and for enzymes which catalyze a color-forming reaction which causes staining of the transformed cell. These can then be selected by automatic cell sorting. Microorganisms which have been successfully transformed with a vector and harbor an appropriate antibiotic resistance gene (for example G418 or hygromycin) can be selected by appropriate antibiotic-containing media or nutrient media. Marker proteins present on the surface of the cell can be used for selection by means of affinity chromatography.

The combination of the host organisms and the vectors appropriate for the organisms, such as plasmids, viruses or phages, such as, for example, plasmids with the RNA polymerase/promoter system, phages λ or μ or other temperate phages or transposons and/or other advantageous regulatory sequences forms an expression system. The term “expression system” means, for example, the combination of mammalian cells, such as CHO cells, and vectors, such as pcDNA3neo vector, which are suitable for mammalian cells.

If desired, the gene product can also be expressed in transgenic organisms such as transgenic animals such as, in particular, mice, sheep or transgenic plants.

Recombinant Production of the Polypeptides:

The invention further relates to methods for the recombinant production of a polypeptide of the invention or functional, biologically active fragments thereof, wherein a polypeptide-producing microorganism is cultured, expression of the polypeptides is induced where appropriate, and they are isolated from the culture. The polypeptides can also be produced on the industrial scale in this way if desired.

The recombinant microorganism can be cultured and fermented by known methods. Bacteria can be grown, for example, in TB or LB medium and at a temperature of 20 to 40° C. and a pH of from 6 to 9. Details of suitable culturing conditions are described, for example, in T. Maniatis, E. F. Fritsch and J. Sambrook, Molecular Cloning: A Laboratory Manual, Cold Spring Harbor Laboratory, Cold Spring Harbor, N.Y. (1989).

If the polypeptides are not secreted into the culture medium, the cells are then disrupted and the product is obtained from the lysate by known protein isolation methods. The cells may alternatively be disrupted by high-frequency ultrasound, by high pressure, such as, for example, in a French pressure cell, by osmolysis, by the action of detergents, lytic enzymes or organic solvents, by homogenizers or by a combination of a plurality of the methods mentioned.

The polypeptides can be purified by known chromatographic methods such as molecular sieve chromatography (gel filtration), such as Q-Sepharose chromatography, ion exchange chromatography and hydrophobic chromatography, and by other usual Die Überstandsfraktion aus beiden Reinigungsverfahren wird einer Chromatographie mit einem geeigneten Harz unterworfen, wobei das gewünschte Molekül mit höherer Selektivität als die Verunreinigungen entweder auf dem Chromatographieharz zurückgehalten wird oder dieses passiert. Diese Chromatographieschritte können nötigenfalls wiederholtwerden, wobei die glei-chen oder andere Chromatographieharze verwendet werden. Der Fachmann ist in der Auswahl der geeigneten Chromatographieharze und ihrer wirksamsten Anwendung für ein bestimmtes zu reinigendes Molekül bewandert. Das gereinigte Produkt kann durch Filtrafion oder Ultrafiltration konzentriert und bei einer Temperatur aufbewahrt werden, bei der die Stabilität des Produktes maximal ist.

Im Stand der Technik sind viele Reinigungsverfahren bekannt. Diese Reinigungstechniken sind z.B. beschrieben in Bailey, J. E. & Ollis, D. F. Biochemical Engineering Fundamentals, McGraw-Hill: New York (1986).

Die Identität und Reinheit der isolierten Verbindungen kann durch Techniken des Standes der Technik bestimmtwerden. Diese umfassen Hochleistungs-Flüssigkeitschromatographie (HPLC), spektroskopische Verfahren, Färbeverfahren, Dünnschichtchromatographie, NIRS, Enzymtest oder mikrobiologische Tests. Diese Analyseverfahren sind zusammengefaβt in: Patek et al. (1994) Appl. Environ. Microbiol. 60: 133-140; Malakhova et al. (1996) Biotekhnologiya 11 27-32; und Schmidt et al. (1998) Bioprocess Engineer. 19: 67-70. Ullmann's Encyclopedia of Industrial Chemistry (1996) Bd. A27, VCH: Weinheim, S. 89-90, S.521-540, S. 540-547, S. 559-566, 575-581 und S.581-587; Michal, G (1999) Biochemical Pathways: An Atlas of Biochemistry and Molecular Biology, John Wiley and Sons; Fallon, A. et al. (1987) Applications of HPLC in Biochemistry in: Laboratory Techniques in Biochemistry and Molecular Biology, Bd. 17.
e) Aligemeine Beschreibung der MPSS-Methode, Klonidentifizierung und Homologiesuche

Die MPSS Technologie (Massive Parallele Signatur Sequenzierung, wie von Brenner et al, Nat Biotechnol.(2000) 18,630-634 beschrieben; worauf hiermit ausdrücklich Bezug genommen wird) wurde an dem filamentösen, Vitamin B2 produzierenden Pilz Ashbya gossypii angewendet Mit Hilfe dieser Technologie ist es möglich, mit hoher Genauigkeit quantitative Aussagen über die Expressionsstärke einer Vielzahl von Genen in einem eukaryotischen Organismus zu erhalten. Dabei wird die mRNA des Organismus zu einem bestimmten Zeitpunkt X isoliert, mit Hilfe des Enzyms Reverse Transkriptase in cDNA umgeschrieben und anschlieβend in spezielle Vektoren kloniert, die eine spezifische Tag-Sequenz besitzen. Die Anzahl von Vektoren mit unterschledlicher Tagsequenz wird dabel so hoch gewählt (etwa 1000-fach höher), dass statistisch gesehen, jedes DNA-Molekül in einen, durch seine Tag-Sequenz einzigartigen, Vektor kioniert wird.

General Experimental Details

a) General Cloning Methods

The cloning steps carried out for the purpose of the present invention, such as, for example, restriction cleavages, agarose gel electrophoresis, purification of DNA fragments, transfer of nucleic acids to nitrocellulose and nylon membranes, linkage of DNA fragments, transformation of E. coli cells, culturing of bacteria, replication of phages and sequence analysis of recombinant DNA, were carried out as described by Sambrook et al. (1989) loc. cit.

b) Polymerase Chain Reaction (PCR)

PCR was carried out in accordance with a standard protocol with the following standard mixture:

8 μl of dNTP mix (200 μM), 10 μl of Taq polymerase buffer (10×) without MgCl₂, 8 μl of MgCl₂ (25 mM), 1 μl of each primer (0.1 μM), 1 μl of DNA to be amplified, 2.5 U of Taq polymerase (MBI Fermentas, Vilnius, Lithuania), demineralized water ad 100 μl.

c) Culturing of E. coli

The recombinant E. coli DH5α strains were cultured in LB-amp medium (tryptone 10.0 g, NaCl 5.0 g, yeast extract 5.0 g, ampicillin 100 g/ml, H₂O ad 1000 ml) at 37° C. For this purpose, in each case one colony was transferred, using an inoculating loop, from an agar plate into 5 ml of LB-amp. After culturing for about 18 hours shaking at a frequency of 220 rpm, 400 ml of medium in a 2 I flask were inoculated with 4 ml of culture. Induction of P450 expression in E. coli took place after the OD578 reached a value between 0.8 and 1.0 by heat-shock induction at 42° C. for three to four hours.

d) Purification of the Required Product From the Culture

The required product can be isolated from the microorganism or from the culture supernatant by various methods known in the art. If the required product is not secreted by the cells, the cells can be harvested from the culture by slow centrifugation, and the cells can be lysed by standard techniques such as mechanical force or ultrasound treatment.

The cell detritus is removed by centrifugation, and the supernatant fraction which contains the soluble proteins is obtained for further purification of the required compound. If the product is secreted by the cells, the cells are removed from the culture by slow centrifugation, and the supernatant fraction is retained for further purification.

The supernatant fraction from the two purification methods is subjected to a chromatography with a suitable resin, with the required molecule either being retained on the chromatography resin, or passing through the latter, with greater selectivity than the impurities. These chromatography steps can be repeated if necessary, using the same or different chromatography resins. The skilled worker is proficient in the selection of suitable chromatography resins and their most effective use for a particular molecule to be purified. The purified product can be concentrated by filtration or ultrafiltration and be stored at a temperature at which the stability of the product is maximal.

Many purification methods are known in the art. These purification techniques are described, for example, in Bailey, J. E. & Ollis, D. F. Biochemical Engineering Fundamentals, McGraw-Hill: New York (1986).

The identity and purity of the isolated compounds can be determined by prior art techniques. These comprise high performance liquid chromatography (HPLC), spectroscopic methods, staining methods, thin layer chromatography, NIRS, enzyme assay or microbiological assays. These analytical methods are summarized in: Patek et al. (1994) Appl. Environ. Microbiol. 60: 133-140; Malakhova et al. (1996) Biotekhnologiya 11 27-32; and Schmidt et al. (1998) Bioprocess Engineer. 19: 67-70. Ullmann's Encyclopedia of Industrial Chemistry (1996) Vol. A27, VCH: Weinheim, pp. 89-90, pp. 521-540, pp. 540-547, pp. 559-566, pp. 575-581 and pp. 581-587; Michal, G (1999) Biochemical Pathways: An Atlas of Biochemistry and Molecular Biology, John Wiley and Sons; Fallon, A. et al. (1987) Applications of HPLC in Biochemistry in: Laboratory Techniques in Biochemistry and Molecular Biology, Vol. 17.

e) General Description of the MPSS Method, Clone Identification and Homology Search

The MPSS technology (Massive Parallel Signature Sequencing as described by Brenner et al, Nat. Biotechnol. (2000) 18, 630-634; to which express reference is hereby made) was applied to the filamentous, vitamin B2-producing fungus Ashbya gossypii. It is possible with the aid of this technology to obtain with high accuracy quantitative information about the level of expression of a large number of genes in a eukaryotic organism. This entails the mRNA of the organism being isolated at a particular time X, being transcribed with the aid of the enzyme reverse transcriptase into cDNA and then being cloned into special vectors which have a specific tag sequence. The number of vectors with a different tag sequence is chosen to be high enough (about 1000 times higher) for statistically each DNA molecule to be cloned into a vector which is unique through its tag sequence.

The vector inserts are then cut out together with the tag. The DNA molecules obtained in this way are then incubated with microbeads which possess the molecular counterparts of the tags mentioned. After incubation it can be assumed that each microbead is loaded via the specific tags or counterparts with only one type of DNA molecules. The beads are transferred into a special flow cell and fixed there so that it is possible to carry out a mass sequencing of all the beads with the aid of an adapted sequencing method based on fluorescent dyes and with the aid of a digital color camera. Although numerically high analysis is possible with this method, it is limited by a reading width of about 16 to 20 base pairs. The sequence length is, however, sufficient to make an unambiguous correlation between sequence and gene possible for most organisms (20 bp have a sequence frequency of ˜1×10¹²; compared with this, the human genome has a size of “only” ˜3×10⁹ bp).

The data obtained in this way are analyzed by counting the number of identical sequences and comparing their frequencies with one another. Frequently occurring sequences reflect a high level of expression, and sequences which occur singly a low level of expression. If the mRNA was isolated at two different time points (X and Y), it is possible to construct a chronological expression pattern of individual genes.

### EXAMPLE 1

Isolation of mRNA from Ashbya gossypii

Ashbya gossypii was cultured in a manner known per se (nutrient medium: 27.5 g/l yeast extract; 0.5 g/l magnesium sulfate; 50 ml/l soybean oil; pH 7). Ashbya gossypii mycelium samples are taken at various times during the fermentation (24 h, 48 h and 72 h), and the corresponding RNA or mRNA is isolated therefrom according to the protocol of Sambrook et al. (1989).

### EXAMPLE 2

Application of the MPSS

Isolated mRNA from A. gossypii is then subjected to an MPSS analysis as explained above.

The sets of data found are subjected to a statistical analysis and categorized according to the significance of the differences in expression. This entailed examination both in relation to an increase and a reduction in the level of expression. A division is made by classifying the change in expression into a) monotonic change, b) change after 24 h, and c) change after 48 h.

The 20 bp sequences representing a change in expression and found by MPSS analysis are then used as probes and hybridized with a gene library from Ashbya gossypii, with an average insert size of about 1 kb. The hybridization temperature in this case was in the range from about 30 to 57° C.

### EXAMPLE 3

Construction of a Genomic Gene Library from Ashbya gossypii

To construct a genomic DNA library, initially chromosomal DNA is isolated by the method of Wright and Philippsen (Gene (1991) 109: 99-105) and Mohr (1995, PhD Thesis, Biozentrum Universität Basel, Switzerland).

The DNA is partially digested with Sau3A. For this purpose, 6 μg of genomic DNA are subjected to a Sau3A digestion with various amounts of enzyme (0.1 to 1 U). The fragments are fractionated in a sucrose density gradient. The 1 kb region is isolated and subjected to a QiaEx extraction. The largest fragments are ligated to the BamHI-cut vector pRS416 (Sikorski and Hieter, Genetics (1988) 122; 19-27) (90 ng of BamHI-cut, dephosphorylated vector; 198 ng of insert DNA; 5 ml of water; 2 μl of 10× ligation buffer; 1 U ligase). This ligation mixture is used to transform the E. coli laboratory strain XL-1 blue, and the resulting clones are employed for identifying the insert.

### EXAMPLE 4

Preparation of an Ordered Gene Library (CHIP Technology)

About 25,000 colonies of the Ashbya gossypii gene library (this corresponds to approximately a 3-fold coverage of the genome) were transferred in an ordered manner to a nylon membrane and then treated by the method of colony hybridization as described in Sambrook et al. (1989). Oligonucleotides were synthesized from the 20 bp sequences found by MPSS analysis and were radiolabeled with ³²P. In each case 10 labeled oligonucleotides with a similar melting point are combined and hybridized together with the nylon membranes. After hybridization and washing steps, positive clones are identified by autoradiography and analyzed directly by PCR sequencing.

In this way, a clone which harbors an insert with the internal name “Oligo 19” and has significant homologies with the MIPS tag “Ygr257c” from S. cerevisiae was identified. The insert has a nucleic acid sequence as shown in SEQ ID NO:1.

In this way, a further clone which harbors an insert with the internal name “Oligo 24” and has significant homologies with the MIPS tag “Mdl2” from S. cerevisiae was identified. The insert has a nucleic acid sequence as shown in SEQ ID NO:5.

In this way, a further clone which harbors an insert with the internal name “Oligo 109” and has significant homologies with the MIPS tag “Prp12” from S. cerevisiae was identified. The insert has a nucleic acid sequence as shown in SEQ ID NO:10.

In this way, a further clone which harbors an insert with the internal name “Oligo 163” and has significant homologies with the MIPS tag “Flx1” from S. cerevisiae was identified. The insert has a nucleic acid sequence as shown in SEQ ID NO:14.

In this way, a further clone which harbors an insert with the internal name “Oligo 31 and has significant homologies with the MIPS tag “STV1” from S. cerevisiae was identified. The insert has a nucleic acid sequence as shown in SEQ ID NO:19.

In this way, a further clone which harbors an insert with the internal name “Oligo 4” and has significant homologies with the MIPS tag “OPT2” from S. cerevisiae was identified. The insert has a nucleic acid sequence as shown in SEQ ID NO:23.

In this way, a further clone which harbors an insert with the internal name “Oligo 6” and has significant homologies with the MIPS tag uVAC1” from S. cerevisiae was identified. The insert has a nucleic acid sequence as shown in SEQ ID NO:28.

In this way, a further clone which harbors an insert with the internal name “Oligo 146” and has significant homologies with the MIPS tag “Ymr162c” from S. cerevisiae was identified. The insert has a nucleic acid sequence as shown in SEQ ID NO:33.

In this way, a further clone which harbors an insert with the internal name “Oligo 56” and has significant homologies with the MIPS tag “Ypl110c” from S. cerevisiae was identified. The insert has a nucleic acid sequence as shown in SEQ ID NO:37.

In this way, a further clone which harbors an insert with the internal name “Oligo 167” and has significant homologies with the MIPS tag “ERP5” from S. cerevisiae was identified. The insert has a nucleic acid sequence as shown in SEQ ID NO:42.

### EXAMPLE 5

Analysis of the Sequence Data by Means of a BLASTX Search

An analysis of the resulting nucleic acid sequences, i.e. their functional assignment to a functional amino acid sequence took place by means of a BLASTX search in sequence databases. Almost all of the amino acid sequence homologies found related to Saccharomyces cerevisiae (baker's yeast). Since this organism had already been completely sequenced, more detailed information about these genes could be referred to under:

http://www.mips.gsf.de/proj/yeast/search/code search.htm.

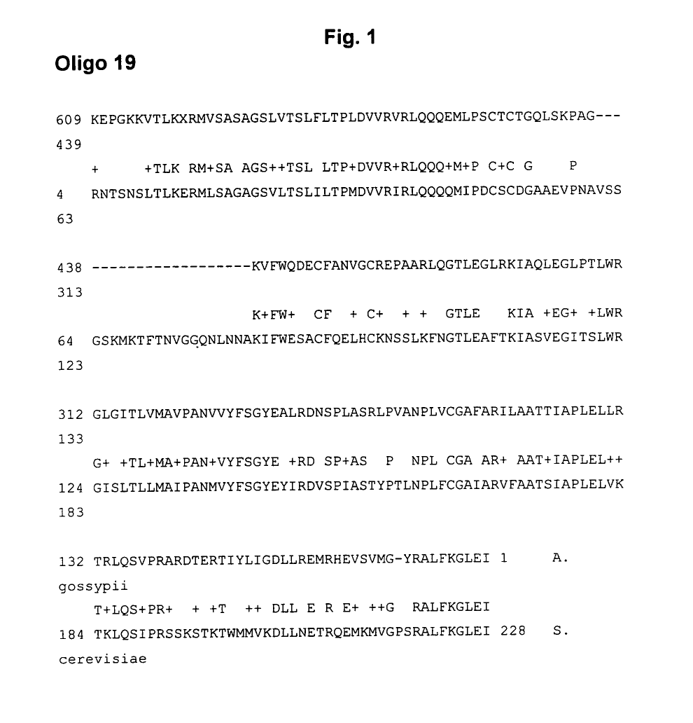

Thus the following homologies with an amino acid fragment from S. cerevisiae were found. The corresponding alignments are shown in FIGS. 1 to 10 which are appended.
a) The amino acid sequence derived from the corresponding complementary strand to SEQ ID NO:1 has significant sequence homology with a mitochondrial energy transfer protein from S. cerevisiae. An amino acid part-sequence derived therefrom (corresponding to nucleotides 609 to 1 from SEQ ID NO: 1) with a part-sequence of the S. cerevisiae protein is depicted in FIG. 1. SEQ ID NO:2 shows an N-terminally extended amino acid part-sequence.

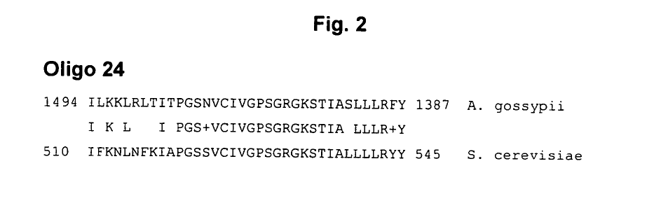

The A. gossypii nucleic acid sequence found could thus be assigned the function of a mitochondrial energy transfer protein.
b) The amino acid sequence derived from the corresponding complementary strand to SEQ ID NO:5 has significant sequence homology with an ABC transport protein from S. cerevisiae. An amino acid part-sequence (SEQ ID NO:6) derived therefrom (corresponding to nucleotides 1494 to 1387 from SEQ ID NO:5) with a part-sequence of the S. cerevisiae protein is depicted in FIG. 2. SEQ ID NO:7 shows a further amino acid part-sequence of the invention.

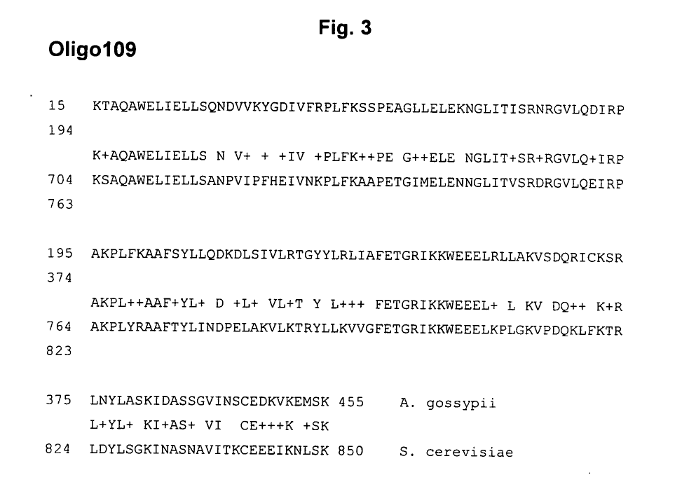

The A. gossypii nucleic acid sequence found could thus be assigned the function of an ABC transport protein.
c) The amino acid sequence derived from the coding strand to SEQ ID NO:10 has significant sequence homology with a membrane-integrated mitochondrial protein from S. cerevisiae. An amino acid part-sequence derived therefrom (corresponding to nucleotides 15 to 455 from SEQ ID NO:10) with a part-sequence of the S. cerevisiae protein is depicted in FIG. 3. SEQ ID NO:11 shows an N-terminally extended amino acid part-sequence.

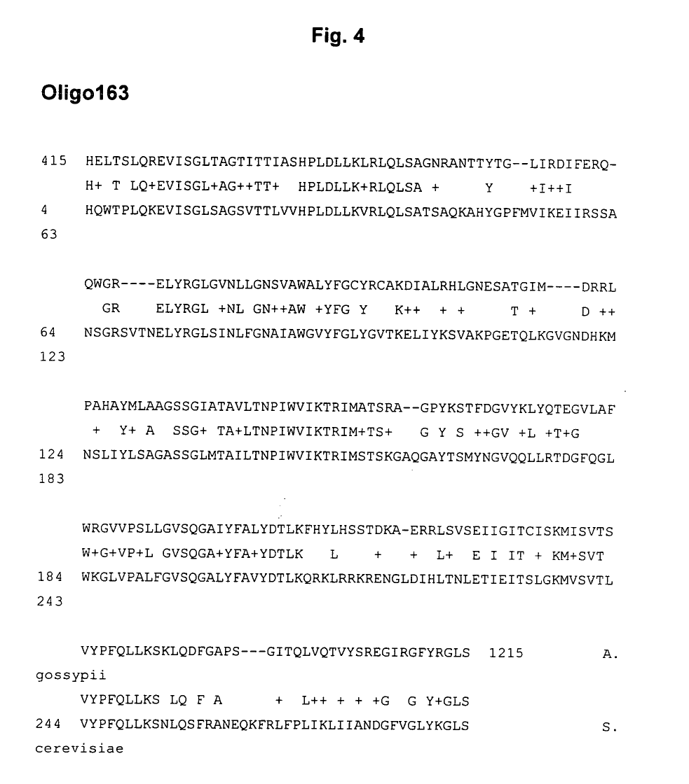

The A. gossypii nucleic acid sequence found could thus be assigned the function of an membrane-integrated mitochondrial protein.
d) The amino acid sequence derived from the coding strand to SEQ ID NO:14 has significant sequence homology with a mitochondrial inner membrane transport protein from S. cerevisiae. An amino acid part-sequence derived therefrom (corresponding to nucleotides 455 to 1215 from SEQ ID NO:14) with a part-sequence of the S. cerevisiae enzyme is depicted in FIG. 4. SEQ ID NO:15 shows an N-terminally extended amino acid part-sequence.

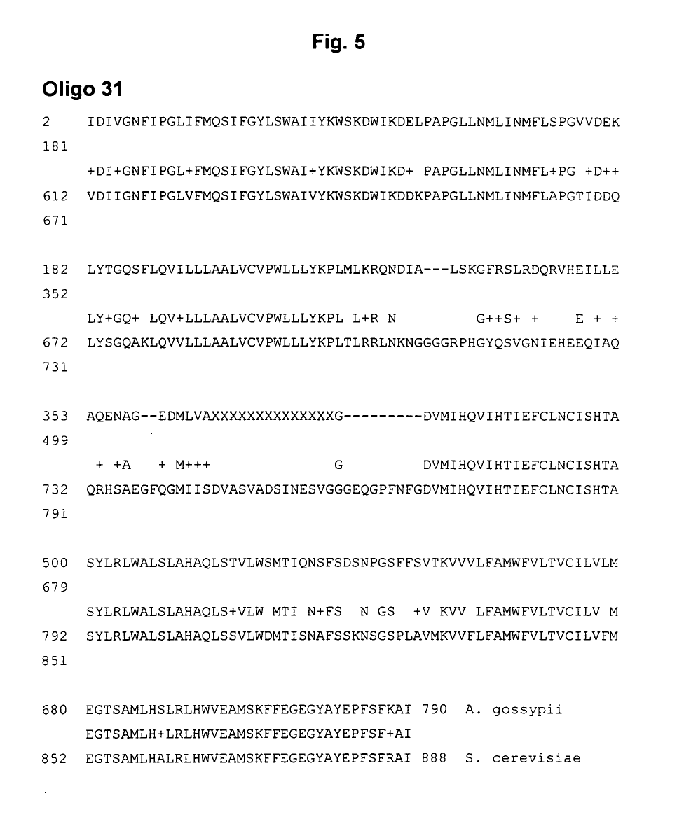

The A. gossypii nucleic acid sequence found could thus be assigned the function of a mitochondrial inner membrane transport protein.
e) The amino acid sequence derived from the coding strand to SEQ ID NO:19 has significant sequence homology with a non-vacuolar 102 kD subunit of the H⁺-ATPase V0 domain from S. cerevisiae. An amino acid part-sequence derived therefrom (corresponding to nucleotides 2 to 790 from SEQ ID NO: 19) with a part-sequence of the S. cerevisiae enzyme is depicted in FIG. 5. SEQ ID NO:20 shows an N-terminally extended amino acid part-sequence.

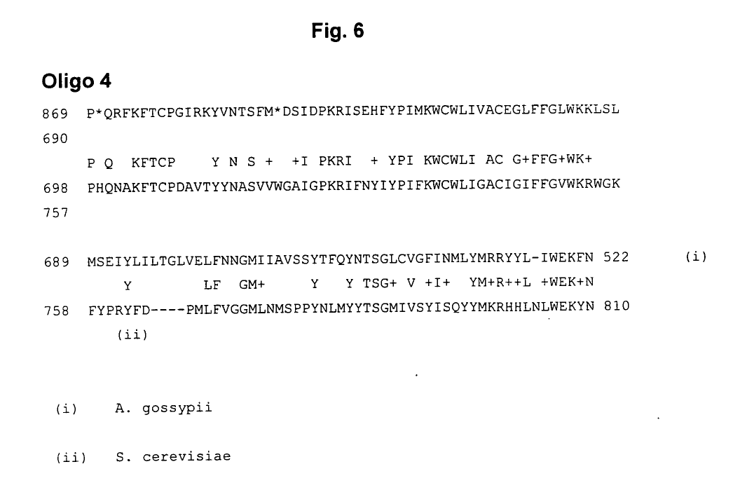

The A. gossypii nucleic acid sequence found could thus be assigned the function of a non-vacuolar 102 kD subunit of the H⁺-ATPase V0 domain.
f) The amino acid sequence derived from the corresponding complementary strand to SEQ ID NO:23 has significant sequence homology with a protein from S. cerevisiae having a similarity to the isp4 protein from S. pombe. An amino acid part-sequence derived therefrom (corresponding to nucleotides 869 to 522 from SEQ ID NO:23) with a part-sequence of the S. cerevisiae enzyme is depicted in FIG. 6. SEQ ID NO:24 shows an N-terminally extended amino acid part-sequence.

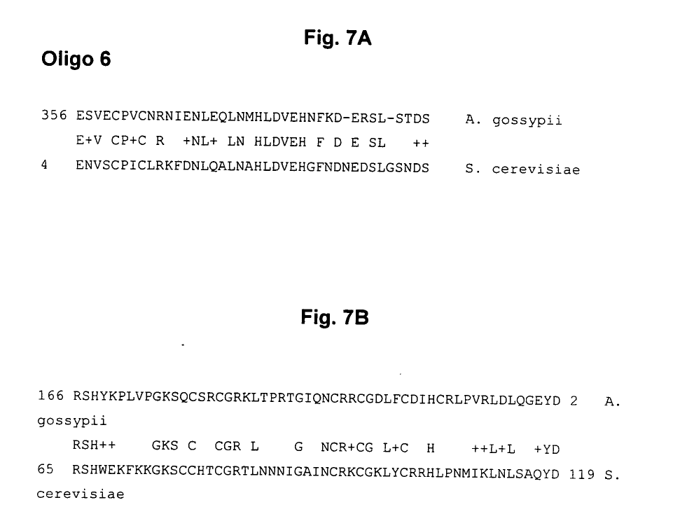

The A. gossypii nucleic acid sequence found could thus be assigned the function of a protein having a similarity with the isp4 protein from S. pombe and thus the activity of an oligopeptide transporter.
g) The amino acid sequence derived from the corresponding complementary strand to SEQ ID NO:28 has significant sequence homology with a VAC1 protein, a cytosolic and peripheral membrane protein having three zinc fingers, from S. cerevisiae. An amino acid part-sequence derived therefrom (corresponding to nucleotides 356 to 243 from SEQ ID NO:28) with a part-sequence of the S. cerevisiae protein is depicted in FIG. 7A. A further amino acid part-sequence derived therefrom (corresponding to nucleotides 166 to 2 from SEQ ID NO:28) with a part-sequence of the S. cerevisiae protein is shown in FIG. 7B. SEQ ID NO: 29 and SEQ ID NO: 30 each show an N-terminally extended amino acid part-sequence.

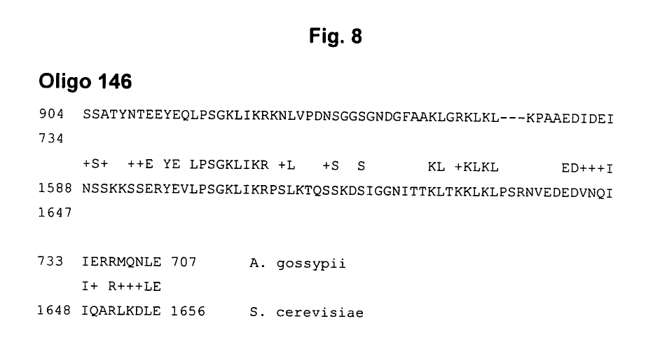

The A. gossypii nucleic acid sequence found could thus be assigned the function of a VAC1 protein, a cytosolic and peripheral membrane protein having three zinc fingers.
h) The amino acid sequence derived from the corresponding complementary strand to SEQ ID NO:33 has significant sequence homology with a protein having an ATPase-like function from S. cerevisiae. An amino acid part-sequence derived therefrom (corresponding to nucleotides 904 to 707 from SEQ ID NO:33) with a part-sequence of the S. cerevisiae enzyme is depicted in FIG. 8. SEQ ID NO:34 shows an N-terminally extended amino acid part-sequence.

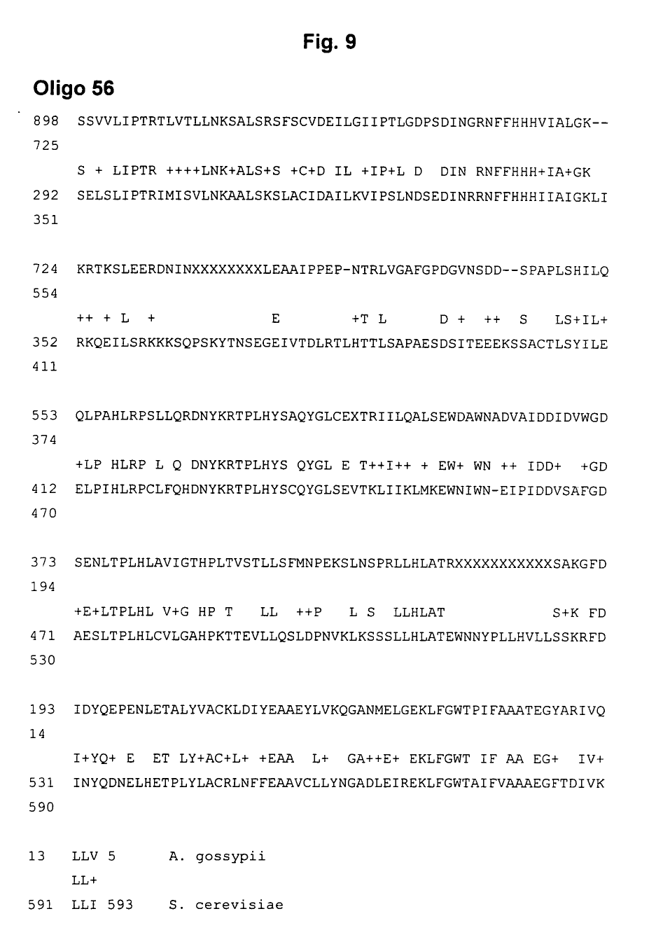

The A. gossypii nucleic acid sequence found could thus be assigned the function of an ATPase-like protein.
i) The amino acid sequence derived from the corresponding complementary strand to SEQ ID NO:37 has significant sequence homology with a PHO85 protein from S. cerevisiae. An amino acid part-sequence derived therefrom (corresponding to nucleotides 898 to 5 from SEQ ID NO:37) with a part-sequence of the S. cerevisiae enzyme is depicted in FIG. 9. The amino acid sequences shown in SEQ ID NO:38 and SEQ ID NO:39 correspond to amino acid part-sequences derived from the complementary strand to position 950 to 900 and 898 to 5, respectively, in SEQ ID NO:37.

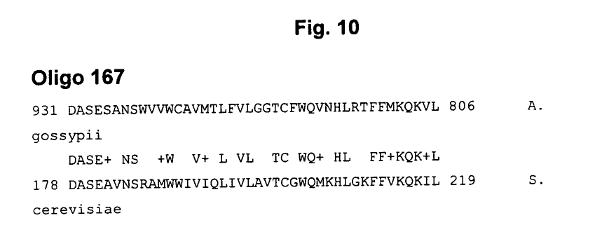

The A. gossypii nucleic acid sequence found could thus be assigned the function of a PHO85 protein.
k) The amino acid sequence derived from the corresponding complementary strand to SEQ ID NO:42 has significant sequence homology with an S. cerevisiae p24 protein involved in membrane trafficking. An amino acid part-sequence derived therefrom (corresponding to nucleotides 931 to 806 from SEQ ID NO:42) with a part-sequence of the S. cerevisiae protein is depicted in FIG. 10. SEQ ID NO:24 shows an N-terminally extended amino acid part-sequence.

The A. gossypii nucleic acid sequence found could thus be assigned the function of a p24 protein involved in membrane trafficking.

### EXAMPLE 6

Isolation of Full-Length DNA

a) Construction of an A. gossypii Gene Library

High molecular weight cellular complete DNA from A. gossypii was prepared from a 2-day old 100 ml culture grown in a liquid MA2 medium (10 g of glucose, 10 g of peptone, 1 g of yeast extract, 0.3 g of myo-inositol ad 1000 ml). The mycelium was filtered off, washed twice with distilled H₂O, suspended in 10 ml of 1 M sorbitol, 20 mM EDTA, containing 20 mg of zymolyase 20T, and incubated at 27° C., shaking gently, for 30 to 60 min. The protoplast suspension was adjusted to 50 mM Tris-HCl, pH 7.5, 150 mM NaCl, 100 mM EDTA and 0.5% strength sodium dodecyl sulfate (SDS) and incubated at 65° C. for 20 min. After two extractions with phenol/chloroform (1:1 vol/vol), the DNA was precipitated with isopropanol, suspended in TE buffer, treated with RNase, reprecipitated with isopropanol and suspended in TE.

An A. gossypii cosmid gene library was produced by binding genomic DNA which had been selected according to size and partially digested with Sau3A to the dephosphorylated arms of the cosmid vector Super-Cos1 (Stratagene). The Super-Cos1 vector was opened between the two cos sites by digestion with Xbal and dephosphorylation with calf intestinal alkaline phosphatase (Boehringer), followed by opening of the cloning site with BamHI. The ligations were carried out in 20 PI, containing 2.5 μg of partially digested chromosomal DNA, 1 μg of Super-Cos1 vector arms, 40 mM Tris-HCl, pH 7.5, 10 mM MgCl₂, 1 mM dithiothreitol, 0.5 mM ATP and 2 Weiss units of T4-DNA ligase (Boehringer) at 15° C. overnight. The ligation products were packaged in vitro using the extracts and the protocol of Stratagene (Gigapack II Packaging Extract). The packaged material was used to infect E. coli NM554 (recA 13, araD139, Δ(ara,leu)7696, Δ(lac)17A, galU, galK, hsrr, rps(str^{r}), mcrA, mcrB) and distributed on LB plates containing ampicillin (50 μg/ml). Transformants containing an A. gossypii insert with an average length of 30-45 kb were obtained.

b) Storage and Screening of the Cosmid Gene Library

In total, 4×10⁴ fresh single colonies were inoculated singly into wells of 96-well microtiter plates (Falcon, No. 3072) in 100 μl of LB medium, supplemented with the freezing medium (36 mM K₂HPO₄/13.2 mM KH₂PO₄, 1.7 mM sodium citrate, 0.4 mM MgSO₄, 6.8 mM (NH₄)₂SO₄, 4.4% (w/v) glycerol) and ampicillin (50 μg/ml), allowed to grow at 37° C. overnight with shaking, and frozen at −70° C. The plates were rapidly thawed and then duplicated in fresh medium using a 96-well replicator which had been sterilized in an ethanol bath with subsequent evaporation of the ethanol on a hot plate. Before the freezing and after the thawing (before any other measures) the plates were briefly shaken in a microtiter shaker (Infors) in order to ensure a homogeneous suspension of cells. A robotic system (Bio-Robotics) with which it is possible to transfer small amounts of liquid from 96 wells of a microtiter plate to nylon membrane (GeneScreen Plus, New England Nuclear) was used to place single clones on nylon membranes. After the culture had been transferred from the 96-well microtiter plates (1920 clones), the membranes were placed on the surface of LB agar with ampicillin (50 μg/ml) in 22×22 cm culture dishes (Nunc) and incubated at 37° C. overnight. Before cell confluence was reached, the membranes were processed as described by Herrmann, B. G., Barlow, D. P. and Lehrach, H. (1987) in Cell 48, pp. 813-825, including as additional treatment after the first denaturation step a 5-minute exposure of the filters to vapors on a pad impregnated with denaturation solution on a boiling water bath.

The random hexamer primer method (Feinberg, A. P. and Vogelstein, B. (1983), Anal. Biochem. 132, pp. 6-13) was used to label double-stranded probes by uptake of [alpha-³²P]dCTP with high specific activity. The membranes were prehybridized and hybridized at 42° C. in 50% (vol/vol) formamide, 600 mM sodium phosphate, pH 7.2, 1 mM EDTA, 10% dextran sulfate, 1% SDS, and 10× Denhardt's solution, containing salmon sperm DNA (50 μg/ml) with ³²P-labeled probes (0.5-1×10⁶ cpm/ml) for 6 to 12 h. Typically, washing steps were carried out at 55 to 65° C. in 13 to 30 mM NaCl, 1.5 to 3 mM sodium citrate, pH 6.3, 0.1% SDS for about 1 h and the filters were autoradiographed at −70° C. with Kodak intensifying screens for 12 to 24 h. To date, individual membranes have been reused successfully more than 20 times. Between the autoradiographies, the filters were stripped by incubation at 95° C. in 2 mM Tris-HCl, pH 8.0, 0.2 mM EDTA, 0.1% SDS for 2×20 min.

c) Recovery of Positive Colonies from the Stored Gene Library

Frozen bacterial cultures in microtiter wells were scraped out using sterile disposable lancets, and the material was streaked onto LB agar Petri dishes containing ampicillin (50 μg/ml). Single colonies were then used to inoculate liquid cultures to produce DNA by the alkaline lysis method (Birnboim, H. C. and Doly, J. (1979), Nucleic Acids Res. 7, pp. 1513-1523).

d) Full-Length DNA

It was possible as described above to identify clones which harbor an insert with the appropriate complete sequence. These clones had the internal names:
“Oligo 19v”. The insert comprising the complete sequence has a nucleic acid sequence as shown in SEQ ID NO:3.
“Oligo 24v”. The insert comprising the complete sequence has a nucleic acid sequence as shown in SEQ ID NO:8.
“Oligo 109v”. The insert comprising the complete sequence has a nucleic acid sequence as shown in SEQ ID NO:12.
“Oligo 163v”. The insert comprising the complete sequence has a nucleic acid sequence as shown in SEQ ID NO:17.
“Oligo 31 v”. The insert comprising the complete sequence has a nucleic acid sequence as shown in SEQ ID NO:21.
“Oligo 4v”. The insert comprising the complete sequence has a nucleic acid sequence as shown in SEQ ID NO:26.
“Oligo 6v”. The insert comprising the complete sequence has a nucleic acid sequence as shown in SEQ ID NO:31.
“Oligo 146v”. The insert comprising the complete sequence has a nucleic acid sequence as shown in SEQ ID NO:35.
“Oligo 56v”. The insert comprising the complete sequence has a nucleic acid sequence as shown in SEQ ID NO:40.
“Oligo 167v”. The insert comprising the complete sequence has a nucleic acid sequence as shown in SEQ ID NO:44.

A survey of all the part-sequences and complete sequences of the invention is to be found in table 1 which follows:

TABLE 1
Sequence survey

| SEQ | | | |
|---|---|---|---|
| ID NO: | Oligo | Description of the sequence | Sequence homology |
| 1 | 019 | DNA part-sequence | mitochondrial energy transfer |
| 2 | 019 | Amino acid part-sequence derived from | protein from S. cerevisiae |
| | | the complementary strand to SEQ ID NO: 1 | |
| 3 | 019 | DNA full-length sequence | |
| 4 | 019 | Amino acid sequence corresponding to | |
| | | the coding region of SEQ ID NO: 3 from | |
| | | position 112 to 294 | |
| 5 | 024 | DNA part-sequence | ABC transport protein from |
| 6 | 024 | Amino acid part-sequence derived from | S. cerevisiae |
| | | the complementary strand to SEQ ID NO: 5 | |
| 7 | 024 | Amino acid part-sequence derived from | |
| | | the complementary strand to SEQ ID NO: 5 | |
| 8 | 024 | DNA full-length sequence | |
| 9 | 024 | Amino acid sequence corresponding to | |
| | | the coding region of SEQ ID NO: 8 from | |
| | | position 820 to 3081 | |
| 10 | 109 | DNA part-sequence | membrane-integrated |
| 11 | 109 | Amino acid part-sequence derived from | mitochondrial protein from |
| | | the coding strand to SEQ ID NO: 10 | S. cerevisiae. |
| 12 | 109 | DNA full-length sequence | |
| 13 | 109 | Amino acid sequence corresponding to | |
| | | the coding region of SEQ ID NO: 12 from | |
| | | position 502 to 2919 | |
| 14 | 163 | DNA part-sequence | Mitochondrial inner membrane |
| 15 | 163 | Amino acid part-sequence derived from | transport protein from S. |
| | | the coding strand to SEQ ID NO: 14 | cerevisiae |
| 16 | 163 | Amino acid part-sequence derived from | |
| | | the coding strand to SEQ ID NO: 14 | |
| 17 | 163 | DNA full-length sequence | |
| 18 | 163 | Amino acid sequence corresponding to | |
| | | the coding region of SEQ ID NO: 17 from | |
| | | position 329 to 1207 | |
| 19 | 31 | DNA part-sequence | non-vacuolar 102 kD subunit of |
| 20 | 31 | Amino acid part-sequence derived from | the H⁺-ATPase V0 domain from |
| | | the coding strand to SEQ ID NO: 19 | S. cerevisiae. |
| 21 | 31 | DNA full-length sequence | |
| 22 | 31 | Amino acid sequence corresponding to | |
| | | the coding region of SEQ ID NO: 21 from | |
| | | position 623 to 3253 | |
| 23 | 4 | DNA part-sequence | isp4 protein from S. pombe |
| 24 | 4 | Amino acid part-sequence derived from | Protein with comparable |
| | | the complementary strand to SEQ ID | function from S. cerevisiae. |
| | | NO: 23 | |
| 25 | 4 | DNA full-length sequence (complementary | |
| | | strand) | |
| 26 | 4 | DNA full-length sequence with ORF region | |
| 27 | 4 | Amino acid sequence corresponding to | |
| | | the coding region of SEQ ID NO: 26 from | |
| | | position 738 to 1037 | |
| 28 | 6 | DNA part-sequence | VAC1 protein, a cytosolic and |
| 29 | 6 | Amino acid part-sequence derived from | peripheral membrane protein |
| | | the complementary strand to SEQ ID | having three zinc fingers, from |
| | | NO: 28 | S. cerevisiae |
| 30 | 6 | Amino acid part-sequence derived from | |
| | | the complementary strand to SEQ ID | |
| | | NO: 28 | |
| 31 | 6 | DNA full-length sequence | |
| 32 | 6 | Amino acid sequence corresponding to | |
| | | the coding region of SEQ ID NO: 31 from | |
| | | position 428 to 1993 | |
| 33 | 146 | DNA part-sequence | Protein with an ATPase or |
| 34 | 146 | Amino acid part-sequence derived from | ATPase-like function from |
| | | the complementary strand to SEQ ID | S. cerevisiae |
| | | NO: 33 | |
| 35 | 146 | DNA full-length sequence | |
| 36 | 146 | Amino acid sequence corresponding to | |
| | | the coding region of SEQ ID NO: 35 from | |
| | | position 537 to 1034 | |
| 37 | 56 | DNA part-sequence | PHO85 protein from |
| 38 | 56 | Amino acid part-sequence derived from | S. cerevisiae |
| | | the complementary strand to SEQ ID | |
| | | NO: 37 | |
| 39 | 56 | Amino acid part-sequence derived from | |
| | | the complementary strand to SEQ ID | |
| | | NO: 37 | |
| 40 | 56 | DNA full-length sequence | |
| 41 | 56 | Amino acid sequence corresponding to | |
| | | the coding region of SEQ ID NO: 40 from | |
| | | position 426 to 4388 | |
| 42 | 167 | DNA part-sequence | p24 protein from S. cerevisiae |
| 43 | 167 | Amino acid part-sequence derived from | |
| | | the complementary strand to SEQ ID | |
| | | NO: 42 | |
| 44 | 167 | Amino acid part-sequence derived from | |
| | | the complementary strand to SEQ ID | |
| | | NO: 42 | |
| 45 | 167 | DNA full-length sequence | |
| 46 | 167 | Amino acid sequence corresponding to | |
| | | the coding region of SEQ ID NO: 45 from | |
| | | position 563 to 1216 | |

1. An isolated polynucleotide derived from a microorganism of Ashbya gossypii that codes for a protein associated with the process of transmembrane transport of said microorganism.

2. The polynucleotide of claim 1, wherein the protein possesses a structural or functional property of a mitochondrial energy transfer protein, an ABC transport protein, a membrane-integrated mitochondrial protein, a mitochondrial inner membrane transport protein, a non-vacuolar 102 kD subunit of an H⁺-ATPase V0 domain, an isp4 protein, a VAC1 protein, a cystolic and peripheral membrane protein having three zinc fingers, a protein with ATPase activity, a protein with an ATPase-like function, a PHO85 protein, or a p24 protein.

3. The polynucleotide of claim 1, comprising the sequence of SEQ ID NO: 1, 5, 10, 14, 19, 23, 28, 33, 37 or 42 a sequence complementary thereto; or a sequence derived from said sequence or said complementary sequence through degeneracy of the genetic code.

4. The polynucleotide of claim 1, which comprises the sequence of SEQ ID NO: 3, 8, 12, 17, 21, 26, 31, 35, 40 or 44, or a fragment thereof.

5. An oligonucleotide that hybridizes with the polynucleotide of claim 1.

6. An isolated nucleic acid that hybridizes with the oligonucleotide of claim 5, and codes for a gene product derived from a microorganism of the genus Ashbya or a functional equivalent thereof.

7. An isolated polypeptide encoded by a the polynucleotide of claim 1 or a fragment thereof.

8. An expression cassette comprising the polynucleotide of claim 1 operatively linked to at least one regulatory nucleic acid sequence.

9. A recombinant vector comprising at least one expression cassette of claim 8.

10. A prokaryotic or eukaryotic host cell transformed with the recombinant vector of claim 9.

11. The host cell of claim 10, wherein functional expression of a gene that codes for said protein is modulated.

12. The host cell of claim 10, which is of the genus Ashbya.

13. A method for microbiological production of vitamin B2 or a precursor or derivative thereof comprising culturing a cell transformed with the vector of claim 9; and
isolating there from the vitamin B2 or the precursor or derivative thereof.

14. A method for recombinant production of the polypeptide of claim 7 comprising culturing a cell transformed with said polynucleotide and isolating said polypeptide there from.

15. A method for detecting an effector target for modulating microbiological production of vitamin B2 or a precursor or derivative thereof, comprising:
treating a microorganism with an effector, wherein said microorganism is capable of the microbiological production of vitamin B2 or the precursor or derivative thereof and wherein said effector target comprises the polypeptide of claim 7 or a nucleic acid sequence that encodes said polypeptide;
detecting an influence of the effector on the effector target by determining a change in the amount of the microbiologically produced vitamin B2 or the precursor or derivative thereof.

16. A method for modulating microbiological production of vitamin B2 or a precursor or derivative thereof, comprising:
treating a microorganism with an effector that interacts with a target,
wherein said microorganism is capable of the microbiological production of vitamin B2 or the precursor or derivative thereof and contains a gene that encodes the polypeptide of claim 7, and
wherein said target is said polypeptide or a nucleic acid sequence that encodes said polypeptide.

17. The method of claim 16, wherein the effector is selected from the group consisting of: antibodies or antigen-binding fragments thereof; polypeptide ligands, which are different from said antibodies or antigen-binding fragments thereof, and interact with the polypeptide; low molecular weight effectors that modulate biological activity of said polypeptide; antisense nucleic acid sequences; ribozymes; and catalytic RNA molecules.

18. A method for microbiological production of vitamin B2 or a precursor or derivative thereof, a comprising:
culturing the host cell of claim 10 in a culture mixture under conditions favoring production of said vitamin B2 or a precursor or derivative thereof; and
isolating a desired product from the culture mixture.

19. The method of claim 18, wherein the host cell is treated with an effector before or during culturing.

20. The method of claim 18, wherein the host cell is a microorganism of the genus Ashbya.

21. The method of claim 18, wherein the desired product is vitamin B2 or a precursor or derivative thereof.

22. A method for modulating production of vitamin B2 or a precursor or derivative thereof of a microorganism of the genus Ashbya comprising:
treating a cell transformed with a polynucleotide with an effector,
wherein said polynucleotide is derived from a microorganism of the genus Ashbya and codes for a protein associated with the process of transmembrane transport in said microorganism, and
wherein the effector modulates the production of vitamin B2 or the precursor or derivative thereof, of said microorganism.

23. A method for modulating transmembrane transport activity of a transmembrane protein or a subsequent state associated therewith in a microorganism of the genus Ashbya comprising:
culturing the microorganism, wherein said microorganism contains a sequence that encodes the polypeptide of claim 7; and
treating said microorganism with an effector that interacts with said polypeptide or said sequence of the microorganism.

24. The host cell of claim 12, which has an improved cellular response to external conditions.

25. The polynucleotide of claim 1, wherein the protein is a transmembrane protein.

26. The polynucleotide of claim 2, wherein the property is derived from a protein of S. cerevisiae.

27. The polynucleotide of claim 2, wherein the property is derived from a protein of S. pombe.

28. The oligonucleotide of claim 5, wherein hybridization is under stringent hybridization conditions.

29. A polypeptide encoded by the polynucleotide of claim 6.

30. A polynucleotide that contains an amino acid sequence comprising at least ten consecutive amino acid residues of SEQ ID NO: 2, 4, 6, 7, 9, 11, 13, 15, 16, 18, 20, 22, 24, 27, 29, 30, 32, 34, 36, 38, 39, 41, 43 or SEQ ID NO: 45; or a functional equivalent thereof.

31. The polynucleotide of claim 30, which possesses a structural or functional property selected from the group consisting of said structural or functional property possessed by a mitochondrial energy transfer protein, an ABC transport protein, a membrane-integrated mitochondrial protein, a mitochondrial inner membrane transport protein, a non-vacuolar 102 kD subunit of an H⁺-ATPase V0 domain, an isp4 protein, a VAC1 protein, a cystolic and peripheral membrane protein having three zinc fingers, a protein with ATPase activity, a protein with an ATPase-like function, a PHO85 protein, and a p24 protein.

32. The host cell of claim 11, wherein the modulation is an increase or a decrease of an activity of said protein expressed by said gene.

33. The method of claim 15, wherein the effector binds to said effector target.

34. The method of claim 15, further comprising isolating said target.

35. The method of claim 23, further comprising isolating vitamin B2 or a precursor or derivative thereof from said culture.

36. The host cell of claim 24, wherein the improved cellular response comprises, as compared to an untransformed cell, a more efficient transmembrane transport, an increased activity of a transmembrane protein, an increased growing and multiplication, an increased viability, an increased yield of a desired product, an increased yield of vitamin B2 or a precursor or derivative there, or a combination thereof.

37. An isolated effector that interacts with an effector target,
wherein the effector is selected from the group consisting of:
antibodies or antigen-binding fragments thereof;
polypeptide ligands that are different from said antibodies or antigen-binding fragments thereof, and that interact with the polypeptide;
low molecular weight effectors that modulate biological activity of a said polypeptide;
antisense nucleic acid sequences;
ribozymes; and
catalytic RNA molecules; and
the effector target is selected from the group consisting of:
a nucleic acid that encodes a polypeptide associated with the process of transmembrane transport of a microorganism of the genus Ashbya; and
a polypeptide encoded by said nucleic acid.

38. The effector of claim 37, wherein the effector target is the nucleic acid and said nucleic acid encodes an amino acid sequence comprising at least ten consecutive amino acid residues of SEQ ID NO: 2, 4, 6, 7, 9, 11, 13, 15, 16, 18, 20, 22, 24, 27, 29, 30, 32, 34, 36, 38, 39, 41, 43 or SEQ ID NO: 45; or a functional equivalent thereof.

39. The effector of claim 37, wherein the effector target is the polypeptide and said polypeptide contains an amino acid sequence comprising at least ten consecutive amino acid residues of SEQ ID NO: 2, 4, 6, 7, 9, 11, 13, 15, 16, 18, 20, 22, 24, 27, 29, 30, 32, 34, 36, 38, 39, 41, 43 or SEQ ID NO: 45; or a functional equivalent thereof.

40. The method of claim 37, wherein the effector binds to said effector target.
